(19) United States
(12) Patent Application Publication
(10) Pub. No.: US 2022/0010007 A1
(43) Pub. Date: Jan. 13, 2022

(54) Treatment for Giant Cell Arteritis

(71) Applicant: Kiniksa Pharmaeuticals, Ltd., Hamilton (BM)

(72) Inventors: John Paolini, Lexington, MA (US); Rohan Gandhi, Lexington, MA (US)

(21) Appl. No.: 17/291,719

(22) PCT Filed: Nov. 7, 2019
(86) PCT No.: PCT/US2019/060255

§ 371 Date: May 6, 2021

Related U.S. Application Data
(60) Provisional application No. 62/758,127, filed on Nov. 9, 2018.
(60) Provisional application No. 62/782,194, filed on Dec. 19, 2018.
(60) Provisional application No. 62/797,813, filed on Jan. 28, 2019.
(60) Provisional application No. 62/883,378, filed on Aug. 6, 2019.

(30) Foreign Application Priority Data
Jul. 30, 2019 (US) PCT/US2019/044231

(51) Int. Cl. C07K 16/24; C07K 16/28; A61K 31/573; A61P 9/00

(52) U.S. Cl. CPC C07K 16/243; A61P 9/00; A61K 31/573; C07K 16/2866

(57) ABSTRACT

The present invention provides, among other things, methods of treating giant cell arteritis, comprising a step of administering to a subject in need of treatment a GM-CSF antagonist (e.g., an anti-GM-CSFRα antibody or an anti-GM-CSF antibody) at a therapeutically effective dose and an administration interval for a treatment period sufficient to improve, stabilize or reduce one or more symptoms of giant cell arteritis relative to a control.

### CROSS-REFERENCE TO RELATED APPLICATIONS

This application claims benefit of, and priority to U.S. Provisional Patent Application Ser. No. 62/883,378 filed on Aug. 6, 2019 and to International Application PCT/US2019/44231 filed on Jul. 30, 2019, which claims priority to U.S. Provisional Application Ser. No. 62/758,127, filed on Nov. 9, 2018; 62/782,194, filed on Dec. 19, 2018; and 62/797,813, filed on Jan. 28, 2019, the contents of each of which are incorporated herein.

### INCORPORATION-BY-REFERENCE OF SEQUENCE LISTING

The contents of the text file named “KPL-034WO2_SL_ST25.txt”, which was created on Nov. 4, 2019 and is 3.77 KB in size, are hereby incorporated by reference in its entirety.

### BACKGROUND

Giant cell arteritis (GCA) is considered the most common form of primary systemic vasculitis. The disease is characterized by inflammation of medium to large blood vessels with predilection for the cranial branches of the carotid artery. Prevalence of this disease in the United States is estimated to be ˜75,000 to 150,000. The risk factors include age, sex, race and geographic region, family history and association with other diseases and health conditions, such as polymyalgia rheumatica. If left untreated, GCA can lead to blindness, may cause aortic aneurism and stroke and can potentially be fatal.

The etiology of the disease is not well known. Current patient care includes placing patients on steroid therapy after suspected diagnosis. The universally accepted course of disease management for GCA is high-dose corticosteroid therapy, usually starting with an oral prednisone dose of 40-60 mg/day. Despite being effective for some patients, many are unable to wean off of corticosteroids because they continue to experience disease flares as the dose is reduced and steroid-sparing treatment options are needed in view of steroid-related complications. There is therefore a high unmet medical need to be addressed in the field.

### SUMMARY OF THE INVENTION

The present invention provides, among other things, methods of treating GCA. In one aspect, the present invention is based on the recent understanding of a role of granulocyte colony stimulating factor in the pathophysiology of the disease. The present invention provides a method for treating GCA, including administering to a subject in need of treatment a composition comprising a granulocyte-macrophage colony-stimulating factor (GM-CSF) antagonist. As used herein, “GM-CSF antagonist” refers to an inhibitor, compound, peptide, polypeptide, protein, or antibody that interacts with GM-CSF or its receptor (GM-CSFR) to reduce or block (either partially or completely) signal transduction that would otherwise result from the binding of GM-CSF to its cognate receptor. In some embodiments, GM-CSF antagonist is an anti-GM-CSF antibody. In some embodiments, GM-CSF antagonist is a granulocyte-macrophage colony-stimulating factor receptor alpha (GM-CSFRα) antagonist. The GM-CSF receptor antagonist is an antibody specific for human GM-CSFRα. The anti-GM-CSFRα antibody is human or humanized antibody.

In some embodiments, the anti-GM-CSFRα antibody is mavrilimumab. The isolation and characterization of mavrilimumab and its variants are described in earlier filings, e.g., WO2007/110631 which is fully incorporated by reference. In some embodiments, the anti-GM-CSFRα antibody comprises a light chain complementary-determining region 1 (LCDR1) defined by SEQ ID NO: 6, a light chain complementary-determining region 2 (LCDR2) defined by SEQ ID NO: 7, and a light chain complementary-determining region 3 (LCDR3) defined by SEQ ID NO: 8; and a heavy chain complementary-determining region 1 (HCDR1) defined by SEQ ID NO: 3, a heavy chain complementary-determining region 2 (HCDR2) defined by SEQ ID NO: 4, and a heavy chain complementary-determining region 3 (HCDR3) defined by SEQ ID NO: 5.

In some embodiments the antibody is a variant of the anti-GM-CSFRα antibody as described in aforementioned patent application. In one embodiment, the anti-GM-CSFRα antibody comprises a light chain variable region having an amino acid sequence at least 90% identical to SEQ ID NO: 2; and a heavy chain variable region having an amino acid sequence at least 90% identical to SEQ ID NO: 1. In one embodiment, the light chain variable region has the amino acid sequence set forth in SEQ ID NO: 2; and the heavy chain variable region has the amino acid sequence set forth in SEQ ID NO: 1.

In one embodiment, the method of the invention treats GCA in a patient population aged between 50 and 85 years of age. In one embodiment, the giant cell arteritis is new-onset disease. In another embodiment, the giant cell arteritis is a relapsing disease. In another embodiment, the giant cell arteritis is a refractory disease.

In some embodiments, the anti-GM-CSFRα antibody is administered concomitantly with other medications including immunomodulatory drugs, such as methotrexate or corticosteroids, and combinations thereof, and optionally weaned from one or more of such concomitant medications following treatment with the anti-GM-CSFRα monoclonal antibody. In one embodiment, the anti-GM-CSFRα antibody therapy is co-administered with corticosteroid. In some embodiments the corticosteroid is prednisone. In some embodiments the subject is administered the anti-GM-CSFRα antibody therapy, along with a steroid taper, that is, the subject is gradually weaned from corticosteroid co-administration after anti-GM-CSFRα monoclonal antibody therapy is initiated. In some embodiments, the successful lowering of or weaning of the subject from steroid co-administration is a measure of efficacy of the anti-GM-CSFRα antibody therapy. In some embodiments, both aspects of (1) lowering or weaning of the subject from steroid co-administration (steroid taper), and (2) maintaining clinical stability of the patient in absence of a recurrence of one or more symptoms is a measure of efficacy of the anti-GM-CSFRα antibody therapy.

In some embodiments, the treating of a subject with anti-GM-CSFRα antibody results in the reduction or amelioration, or slowing or halting progression of at least one of the disease symptoms associated with GCA. In some embodiments, the treating results in prevention of the disease symptoms associated with GCA. The symptoms associated with GCA comprise fever, fatigue, weight loss, headache, temporal tenderness, and jaw claudication; transient monocular visual loss (TMVL) and anterior ischemic optic neuropathy (AION), aortic aneurism and vasculitis. In one embodiment, a biomarker for the disease is serum inflammatory marker CRP ≥1 mg/dL. In one embodiment, a biomarker for the disease is ESR ≥30 mm/hour. In one embodiment, the administration of anti-GM-CSFRα antibody results in lowering of serum inflammatory marker CRP <1 mg/dL and/or ESR ≤30 mm/hour. In one embodiment, the administration of anti-GM-CSFRα antibody results in sustained lowering of serum inflammatory marker CRP <1 mg/dL and/or ESR ≤30 mm/hour for 26 weeks or more.

In some embodiments, the treating results in elimination of symptoms associated with GCA. In some embodiments, the treating reduces arterial inflammation and/or reduces expression of genes associated with GCA lesions. In some embodiments, the reduced expression of genes associated with GCA lesions results in reduced expression of protein and/or messenger RNA (mRNA) selected from GM-CSF, GM-CSFRα, JAK2, IL-6, CD83, PU.1, HLA-DRA, CD3E, TNFα, IL-1β, or combinations thereof. Accordingly, in some embodiments, the treating reduces expression of GM-CSF. In some embodiments, the treating reduces expression of GM-CSFRα. In some embodiments, the treating reduces expression of JAK2. In some embodiments, the treating reduces expression of IL-6. In some embodiments, the treating reduces expression of CD83. In some embodiments, the treating reduces expression of PU.1. In some embodiments, the treating reduces expression of HLA-DRA. In some embodiments, the treating reduces expression of CD3E. In some embodiments, the treating reduces expression of TNFα. In some embodiments, the treating reduces expression of IL-1β.

In some embodiments, the treating results in the reduction or elimination of infiltrated macrophages, reduced T-cells in vessel adventitia, reduced GM-CSFRα expression in vasa vasorum of the temporal artery, reduced density of inflammatory infiltrates, and/or reduced or stabilized vessel wall remodeling. In some embodiments, the treating results in a reduction of cells positive for GM-CSF or INF-γ in the arterial wall. In some embodiments, the treating results in a reduction of cells positive for GM-CSF in the arterial wall. In some embodiments, the treating results in a reduction of cells positive for INF-γ in the arterial wall. In some embodiments, the treating results in a reduction of cells positive for GM-CSF and INF-γ in the arterial wall.

In some embodiments, the treating normalizes gene expression levels comparable to a subject who does not have GCA. In some embodiments, the treating normalizes gene expression levels of genes associated with interferon signaling, IL-6 signaling and/or GM-CSF signaling. In some embodiments, the treating normalizes gene expression levels of genes associated with interferon signaling selected from INF-γ, INF-αR1, INF-γR1, INF-γR2, IFI30, IFI35, PRKCD, B2M, IFNAR1, CIITA, PTPN2, PTPN11, IRF1, IFR5, IRF8, GBP1, GBP5, STAT1, STAT2, FCγR1A/B, ICAM1, VCAM1, TYK2, CD44, IP6K2, DDX58, PTPN6, or combinations thereof. In some embodiments, the treating normalizes gene expression levels of genes associated with IL-6 signaling selected from PTPN11, TYK2, STAT1, IL-11RA, IL-6, or combinations thereof. In some embodiments, the treating normalizes gene expression levels of genes associated with GM-CSF signaling selected from IL-2RB, IL-2RG, GM-CSFRα, JAK3, STAT5A, SYK, PTPN11, HCK, FYN, INPP5D, BLNK, PTPN6, or combinations thereof.

In some embodiments, the dose of the co-administered corticosteroid is tapered over the course of the treatment with the GM-CSF antagonist. In some embodiments, the steroid taper is spread over a period of 26 weeks. In some embodiments, the steroid taper is spread over a period of 52 weeks. In some embodiments, the steroid taper is spread over a period of any period between 26 weeks and 52 weeks.

In one embodiment, the composition comprising anti-GM-CSFRα antibody is administered at a dose of about 150 mg. In some embodiments, the composition comprising anti-GM-CSFRα is administered at a dose of 150 mg. In some embodiments, the composition comprising anti-GM-CSFRα antibody is administered subcutaneously. In some embodiments, the composition comprising anti-GC-CSFRα antibody is administered intravenously. In some embodiments the composition comprising anti-GM-CSFRα antibody is administered once every two weeks. In some embodiments the composition comprising anti-GM-CSFRα antibody is administered once every week. In some embodiments, mavrilimumab is administered once a week by intravenous or subcutaneous administration at a dose of 150 mg. In some embodiments, mavrilimumab is administered once every two weeks by intravenous or subcutaneous administration at a dose of 150 mg.

In some embodiments, the therapeutically effective dose of an anti-GM-CSFRα antibody for treating GCA is equal to or greater than 0.1 mg/kg, 0.3 mg/kg, 0.5 mg/kg, 0.7 mg/kg, 1 mg/kg, 1.25 mg/kg, 1.5 mg/kg, 1.75 mg/kg, 2 mg/kg, 5 mg/kg, 7.5 mg/kg, or 10 mg/kg.

In some embodiments, the therapeutically effective dose of 0.5-2.5 mg/kg is delivered by subcutaneous administration.

In some embodiments, the therapeutically effective dose is administered once a week. In some embodiments, the therapeutically effective dose is administered twice a week. In some embodiments, the therapeutically effective dose is administered once every two weeks.

In some embodiments, the subject is co-administered an additional therapeutic agent. In some embodiments, the additional therapeutic agent is a corticosteroid. In some embodiments, the corticosteroid is prednisone. In some embodiments, the additional therapeutic is a co-administered corticosteroid that is tapered over 26 weeks.

In some embodiments, administering the composition comprising anti-GM-CSFRα antibody reduces serum inflammatory marker CRP to <1 mg/dL. In some embodiments, administering the composition comprising anti-GM-CSFRα antibody reduces ESR ≤30 mm/hour. In some embodiments, administering the composition comprising anti-GM-CSFRα antibody results in sustained remission of symptoms associated with GCA. In some embodiments, administering the composition comprising anti-GM-CSFRα antibody results in patients achieving a sustained remission of symptoms associated with GCA for about 26 weeks. In some embodiments, administering the composition comprising anti-GM-CSFRα antibody results in patients achieving a sustained remission of symptoms associated with GCA for 26 weeks.

In some embodiments, the remission is sustained with a reduction of co-administered corticosteroids. In some embodiments, the sustained remission is substantially corticosteroid-free. In some embodiments, the sustained remission is corticosteroid free.

It is to be understood that all embodiments as described above are applicable to all aspects of the present invention.

### BRIEF DESCRIPTION OF THE DRAWING

The drawings are for illustration purposes only not for limitation.

FIG. 1 is a graph that illustrates a GCA treatment algorithm currently followed by medical practitioners.

FIG. 2 is a graphical illustration that depicts a GCA clinical study design described herein using the anti-GM-CSFRα antibody (designated as Antibody in the graphical illustration) described in Example 1.

FIG. 3 depicts a graphical illustration of the design of a phase 2, randomized, double blind, placebo-controlled multi-center clinical study for efficacy and safety of using the anti-GM-CSFRα antibody (designated as Antibody in the graphical illustration) in GCA patients.

FIG. 4 depicts mRNA expression levels of Pu.1 mRNA relative to that of a housekeeping gene in cultured temporal artery biopsies from subjects having giant cell arteritis (GCA+) or control subjects with no giant cell arteritis (Controls).

FIG. 5 depicts mRNA expression levels of CD83 mRNA relative to that of a housekeeping gene in cultured temporal artery biopsies from subjects having giant cell arteritis (GCA+) or control subjects with no giant cell arteritis (Controls).

FIGS. 6A and 6B depict graphs that show selected gene expression levels obtained from temporal arteries of subjects who have GCA in comparison to temporal arteries obtained from subjects who do not have GCA. The data show that expression of GM-CSF- and T_{H}1-associated genes is increased in subjects who have GCA (shaded bars) compared to subjects who do not have GCA (open bars).

FIGS. 7A and 7B depict the mRNA expression levels of GM-CSF (FIG. 7A) and GM-CSF-receptor alpha (GM-CSFRα) (FIG. 7B) relative to that of a housekeeping gene GUSb in cultured temporal artery biopsies from subjects having giant cell arteritis (GCA) or control subjects with no giant cell arteritis (Controls). FIG. 7C depicts the mRNA expression levels of interferon-γ relative to that of a housekeeping gene GUSb in fresh temporal artery biopsies from subjects having giant cell arteritis (GCA+) or control subjects with no giant cell arteritis (Controls).

FIG. 8A is a generalized schematic that shows the temporal artery culture model that was used to assess the effect of mavrilimumab on gene expression of arteries obtained from GCA patients in comparison to subjects that do not have GCA. FIG. 8B shows data obtained from cultured temporal arteries from subjects with GCA that were exposed to either mavrilimumab or placebo. For both GCA and control arteries, each vessel was divided into two sections; one section was treated with mavrilimumab and the other section was treated with placebo. FIG. 8B shows that culturing GCA arteries with mavrilimumab results in a decrease in the expression of CD83, PU.1, HLA-DRA, CD3ε, TNFα, and CXCL10. Data points derived from the same patient sample are connected by lines.

FIG. 9A shows immunohistochemistry (IHC) staining for CD3⁺ T-cells in the inflamed, grafted human arteries treated in vivo with IgG control antibody or anti-GM-CSFRα antibody.

FIG. 9B depicts a graph that shows density of the T-cell infiltrates measured by enumeration of CD3⁺ cells per high-powered field (HPF).

FIG. 10 is a graph quantifying the number of microvessels and the intimal layer thickness in inflamed arteries treated with IgG control antibody or anti-GM-CSFRα antibody.

FIG. 11 is a gene expression heatmap from inflamed arteries treated with IgG control antibody or anti-GM-CSFRα antibody. Each row represents gene and each column represents a mouse. The expression level is scaled from 0 to 4. “ns” indicates not significant.

### DEFINITIONS

In order for the present invention to be more readily understood, certain terms are first defined below. Additional definitions for the following terms and other terms are set forth throughout the specification. The publications and other reference materials referenced herein to describe the background of the invention and to provide additional detail regarding its practice are hereby incorporated by reference.

Amino acid: As used herein, term “amino acid,” in its broadest sense, refers to any compound and/or substance that can be incorporated into a polypeptide chain. In some embodiments, an amino acid has the general structure H₂N—C(H)(R)—COOH. In some embodiments, an amino acid is a naturally occurring amino acid. In some embodiments, an amino acid is a synthetic amino acid; in some embodiments, an amino acid is a d-amino acid; in some embodiments, an amino acid is an l-amino acid. “Standard amino acid” refers to any of the twenty standard 1-amino acids commonly found in naturally occurring peptides. “Nonstandard amino acid” refers to any amino acid, other than the standard amino acids, regardless of whether it is prepared synthetically or obtained from a natural source. As used herein, “synthetic amino acid” encompasses chemically modified amino acids, including but not limited to salts, amino acid derivatives (such as amides), and/or substitutions. Amino acids, including carboxyl- and/or amino-terminal amino acids in peptides, can be modified by methylation, amidation, acetylation, protecting groups, and/or substitution with other chemical groups that can change the peptide's circulating half-life without adversely affecting their activity. Amino acids may participate in a disulfide bond. Amino acids may comprise one or posttranslational modifications, such as association with one or more chemical entities (e.g., methyl groups, acetate groups, acetyl groups, phosphate groups, formyl moieties, isoprenoid groups, sulfate groups, polyethylene glycol moieties, lipid moieties, carbohydrate moieties, biotin moieties, etc.). The term “amino acid” is used interchangeably with “amino acid residue,” and may refer to a free amino acid and/or to an amino acid residue of a peptide. It will be apparent from the context in which the term is used whether it refers to a free amino acid or a residue of a peptide.

Amelioration: As used herein, the term “amelioration” is meant the prevention, reduction or palliation of a state, or improvement of the state of a subject. Amelioration includes, but does not require complete recovery or complete prevention of a disease condition. In some embodiments, amelioration includes increasing levels of relevant protein or its activity that is deficient in relevant disease tissues.

Approximately or about: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Delivery: As used herein, the term “delivery” encompasses both local and systemic delivery.

Half-life: As used herein, the term “half-life” is the time required for a quantity such as nucleic acid or protein concentration or activity to fall to half of its value as measured at the beginning of a time period.

Improve, increase, or reduce: As used herein, the terms “improve,” “increase” or “reduce,” or grammatical equivalents, indicate values that are relative to a baseline measurement, such as a measurement in the same individual prior to initiation of the treatment described herein, or a measurement in a control subject (or multiple control subject) in the absence of the treatment described herein, e.g., a subject who is administered a placebo. A “control subject” is a subject afflicted with the same form of disease as the subject being treated, who is about the same age as the subject being treated.

Neutralization: As used herein, neutralization means reduction or inhibition of biological activity of the protein to which the neutralizing antibody binds, in this case GM-CSF or GM-CSFR, e.g. reduction or inhibition of GM-CSF binding to GM-CSFRα, or of signaling by GM-CSFRα e.g. as measured by GM-CSFRα-mediated responses. The reduction or inhibition in biological activity may be partial or total. The degree to which an antibody neutralizes GM-CSF or GM-CSFR is referred to as its neutralizing potency.

Patient: As used herein, the term “patient” refers to any organism to which a provided composition may be administered, e.g., for experimental, diagnostic, prophylactic, cosmetic, and/or therapeutic purposes. Typical patients include animals (e.g., mammals such as mice, rats, rabbits, non-human primates, and/or humans). In some embodiments, a patient is a human. A human includes pre- and post-natal forms.

Pharmaceutically acceptable: The term “pharmaceutically acceptable” as used herein, refers to substances that, within the scope of sound medical judgment, are suitable for use in contact with the tissues of human beings and animals without excessive toxicity, irritation, allergic response, or other problem or complication, commensurate with a reasonable benefit/risk ratio.

Substantial identity: The phrase “substantial identity” is used herein to refer to a comparison between amino acid or nucleic acid sequences. As will be appreciated by those of ordinary skill in the art, two sequences are generally considered to be “substantially identical” if they contain identical residues in corresponding positions. As is well known in this art, amino acid or nucleic acid sequences may be compared using any of a variety of algorithms, including those available in commercial computer programs such as BLAS TN for nucleotide sequences and BLASTP, gapped BLAST, and PSI-BLAST for amino acid sequences. Exemplary such programs are described in Altschul, et al., Basic local alignment search tool, J Mal. Biol., 215(3): 403-410, 1990; Altschul, et al., Methods in Enzymology; Altschul et al., Nucleic Acids Res. 25:3389-3402, 1997; Baxevanis et al., Bioinformatics: A Practical Guide to the Analysis of Genes and Proteins, Wiley, 1998; and Misener, et al., (eds.), Bioinformatics Methods and Protocols (Methods in Molecular Biology, Vol. 132), Humana Press, 1999. In addition to identifying identical sequences, the programs mentioned above typically provide an indication of the degree of identity. In some embodiments, two sequences are considered to be substantially identical if at least 50%, 55%, 60%, 65%, 70%, 75%, 80%, 85%, 90%, 91%, 92%, 93%, 94%, 95%, 96%, 97%, 98%, 99% or more of their corresponding residues are identical over a relevant stretch of residues. In some embodiments, the relevant stretch is a complete sequence. In some embodiments, the relevant stretch is at least 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500 or more residues.

Suitable for subcutaneous delivery: As used herein, the phrase “suitable for subcutaneous delivery” or “formulation for subcutaneous delivery” as it relates to the pharmaceutical compositions of the present invention generally refers to the stability, viscosity, tolerability and solubility properties of such compositions, as well as the ability of such compositions to deliver an effective amount of antibody contained therein to the targeted site of delivery.

Subject: As used herein, the term “subject” refers to a human or any non-human animal (e.g., mouse, rat, rabbit, dog, cat, cattle, swine, sheep, horse or primate). A human includes pre- and post-natal forms. In many embodiments, a subject is a human being. A subject can be a patient, which refers to a human presenting to a medical provider for diagnosis or treatment of a disease. The term “subject” is used herein interchangeably with “individual” or “patient.” A subject can be afflicted with or is susceptible to a disease or disorder but may or may not display symptoms of the disease or disorder.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Systemic distribution or delivery: As used herein, the terms “systemic distribution,” “systemic delivery,” or grammatical equivalent, refer to a delivery or distribution mechanism or approach that affect the entire body or an entire organism. Typically, systemic distribution or delivery is accomplished via body's circulation system, e.g., blood stream. Compared to the definition of “local distribution or delivery.”

Target tissues: As used herein, the term “target tissues” refers to any tissue that is affected by a disease or disorder to be treated. In some embodiments, target tissues include those tissues that display disease-associated pathology, symptom, or feature.

Therapeutically effective amount: As used herein, the term “therapeutically effective amount” of a therapeutic agent means an amount that is sufficient, when administered to a subject suffering from or susceptible to a disease, disorder, and/or condition, to treat, diagnose, prevent, and/or delay the onset of the symptom(s) of the disease, disorder, and/or condition. It will be appreciated by those of ordinary skill in the art that a therapeutically effective amount is typically administered via a dosing regimen comprising at least one unit dose.

Treating: As used herein, the term “treat,” “treatment,” or “treating” refers to any method used to partially or completely alleviate, ameliorate, relieve, inhibit, prevent, delay onset of, reduce severity of and/or reduce incidence of one or more symptoms or features of a particular disease, disorder, and/or condition. Treatment may be administered to a subject who does not exhibit signs of a disease and/or exhibits only early signs of the disease for the purpose of decreasing the risk of developing pathology associated with the disease.

### DETAILED DESCRIPTION

The present invention provides, among other things, methods of treating giant cell arteritis (GCA). The method comprises a step of administering to a subject in need of treatment a GM-CSF antagonist (e.g., an anti-GM-CSFRα or anti-GM-CSF antibody) at a therapeutically effective dose and an administration interval for a treatment period sufficient to improve, stabilize or reduce one or more symptoms of GCA relative to a control. A control as used in the context of this administration is the state of the symptoms at a time prior to the administration of the antibody.

Various aspects of the invention are described in detail in the following sections. The use of sections is not meant to limit the invention. Each section can apply to any aspect of the invention. In this application, the use of “or” means “and/or” unless stated otherwise.

### Giant Cell Arteritis

Giant cell arteritis (GCA) is an auto-inflammatory/auto-immune disease that targets life-sustaining tissues, specifically the blood vessels. Abnormal immune response driven by T cells and macrophages lead to destruction of the vessel wall and induce maladaptive repair mechanisms that eventually cause vessel occlusion and organ ischemia. Pathological manifestations occur in the aorta and its 2nd-5th branches, including vessels supplying the optic nerve. GCA is characterized by blood vessel inflammation and infiltration of monocytes, macrophages and the aggregation into giant cells, which are multinucleated fusions of macrophages. It is an inflammatory disease of large and medium-sized arteries that causes headaches, ischemic visual loss, and jaw and other muscle claudication (Dejaco C et al., Nat Rev Rheumatol. 2017, 13(10):578-592). If left untreated, GCA can lead to monocular or binocular blindness, aortic aneurysm, myocardial infarction, and, rarely, stroke and death (Weyand C M, and Goronzy J J., N Engl J Med. 2014, 371(1):50-7). GCA presents with a wide and variable spectrum of signs and symptoms (Weyand and Goronzy, 2014). The early clinical signs and symptoms include new onset of headaches, abrupt onset of visual disturbances, jaw claudication, fever, fatigue, weight loss, transient monocular visual loss (TMVL) and anterior ischemic optic neuropathy (AION). Diagnosis is usually made provisionally on the basis of clinical signs and symptoms and then confirmed by color Doppler ultrasound (CDUS) or by temporal artery biopsy (TAB) (Dejaco C, et al. Ann Rheum Dis. 2018 Jan. 22. doi: 10.1136/annrheumdis-2017-212649). In the United States (US), the lifetime risk of developing GCA has been estimated at approximately one percent in women and 0.5 percent in men (Crowson C S et al., Arthritis Rheum. 2011 March, 63(3):633-9). GCA generally affects adults over 50 years of age, with a 3:1 imbalance of women to men (Weyand and Goronzy, 2014). The reported prevalence of proven GCA in populations aged over 50 years varies significantly geographically and ranges between 24-200 per 100,000 individuals in the European Union (EU) and 24-278 per 100,000 individuals in the US (Salvarani C et al. Arthritis Rheum 2004, 51:264-8; Lawrence R C et al. Arthritis Rheum. 2008, 58:26-35; Lee J I et al. Clinic Rev Allergy Immunol 2008, 35: 88-95).

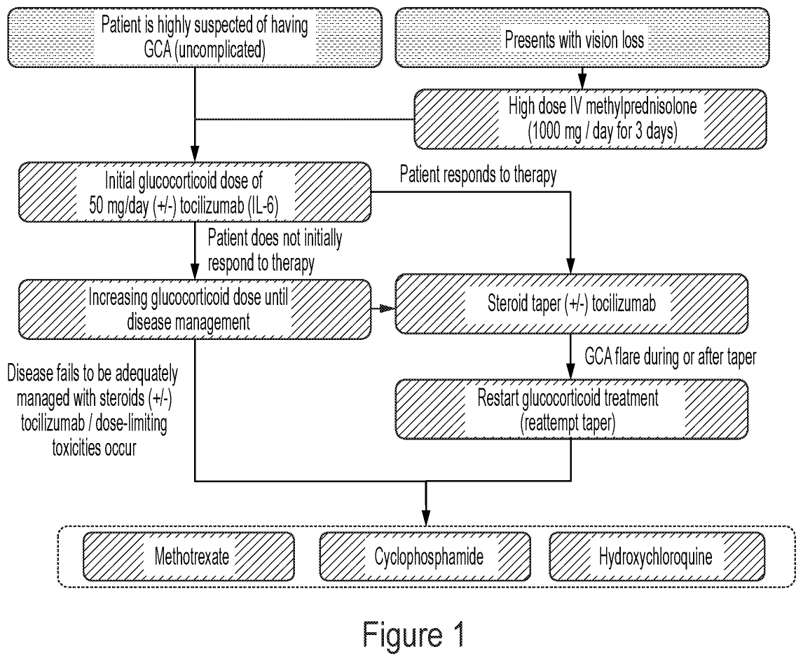

The current treatment modalities include administration of steroids upon diagnosis of GCA in a patient. FIG. 1 illustrates a current GCA treatment algorithm followed by practitioners with patients who present an uncomplicated disease scenario (left side of FIG. 1), and an algorithm followed when a patient presents with advanced symptoms, for example, with vision loss (right side of FIG. 1). Glucocorticoids are the mainstay of treatment because they normalize inflammatory markers. In general, a high response to steroid therapy is noticed in the majority of patients, with improvements evident within the first few days of therapy. However, many patients receive long courses of this therapy to prevent disease flare-up, and long-term use is associated with significant and serious side effects, including glaucoma, fluid retention, hypertension, mood changes, memory changes, other psychological effects, weight gain, and diabetes (Roberts J, and Clifford A, Ther Adv Chronic Dis. 2017 April, 8(4-5):69-7). Significant proportions (˜50%) of patients suffer from disease relapses or more chronic disease and require high doses of prednisone for a number of years to control symptoms. While effective for some patients, many times patients are unable to wean from corticosteroids because they continue to experience disease flares as the dose is reduced (Dejaco et al, 2017; Salvarani et al, 2012) (Deng et al., Circulation. 2010 Feb. 23; 121(7): 906-915). In one study cohort that followed 106 patients with GCS over 4.5 to 10.1 years, 68 patients (64%) experienced at least one relapse, and 38 (36%) experienced two or more relapses during or after corticosteroid weaning (Alba M A, et al. Medicine (Baltimore). 2014; 93(5):194-201). Studies suggest that a subset of patients continue to develop visual symptoms despite long-term, high dose steroid therapy. According to another study, temporal arterial biopsy results were positive for arteritis in 31% of patients (89 of 286) who did not receive corticosteroids before biopsy and for 35% of those (86 of 249) who did receive corticosteroids before biopsy (P=0.4; 95% confidence interval for the difference, −4.7% to 11.5%) (Achkar et al. Ann Intern Med. 1994 Jun. 15; 120 (12):987-92). These data suggest that steroids do not affect the underlying disease process in all patients.

The etiopathology of the disease was not well understood for a long time, principally because of the paucity of information on the mechanism of blood vessel wall damage. However, clues to the pathogenic events may derive from understanding the functioning of the tissue infiltrating cells. Arterial injury in GCA is associated with the formation of granulomas that are composed largely of activated macrophages, infiltrated T cells, such that the vascular lesions are found to be T-cell dependent. Experimental evidence in SCID mice suggests that glucocorticoid treatment inhibits the T-cell mediated pathology but does not adequately suppress tissue-infiltrating macrophage function. Macrophages constitute a key cell type generated and maintained by GM-CSF signaling, and thus may explain why many patients require long-term chronic treatment and are unable to wean off corticosteroids (Brack A et al. J Clin Invest. 1997, 99(12):2842-50). Blocking GM-CSF signaling at the receptor can provide additional benefit to these patients by reducing long-term sequelae that result from chronic vessel inflammation and reducing steroid dependency. ACTEMRA® (tocilizumab), an interleukin-6 receptor inhibitor, recently received marketing approval in the US and Europe in GCA for use concomitantly with a corticosteroid taper; however, just under 50% of patients did not achieve sustained remission over 52 weeks on tocilizumab after a 26-week corticosteroid taper (Stone J H et al. N Engl J Med. 2017; 377(15):1494-1495). Accordingly, there is still an unmet need for improved therapeutic options for treating patients with GCA.

### GM-CSF Biology

GM-CSF is a type I proinflammatory cytokine which enhances survival and proliferation of a broad range of hematopoietic cell types. It is a growth factor first identified as an inducer of differentiation and proliferation of myeloid cells (e.g., neutrophils, basophils, eosinophils, monocytes, and macrophages) (Wicks I P and Roberts A W. Nat Rev Rheumatol. 2016, 12(1):37-48). Studies using different approaches have demonstrated that with GM-CSF overexpression, pathological changes almost always follow (Hamilton J A et al., Growth Factors. 2004, 22(4):225-31). GM-CSF enhances trafficking of myeloid cells through activated endothelium of blood vessels and can also contribute to monocyte and macrophage accumulation in blood vessels during inflammation. GM-CSF also promotes activation, differentiation, survival, and proliferation of monocytes and macrophages as well as resident tissue macrophages in inflamed tissues. It regulates the phenotype of antigen-presenting cells in inflamed tissues by promoting the differentiation of infiltrating monocytes into M1 macrophages and monocyte-derived dendritic cells (MoDCs). Moreover, the production of IL-23 by macrophages and MoDCs, in combination with other cytokines such as IL-6 and IL-1, modulates T-cell differentiation.

Together with M-CSF (macrophage-colony stimulating factor), GM-CSF regulates the number and function of macrophages that turn into histiocytic and multinucleated giant cells, the key effector cells in the vasculitic lesions of GCA. Macrophages activated by GM-CSF acquire a series of effector functions, all of which identify them as inflammatory macrophages. GM-CSF-activated macrophages produce proinflammatory cytokines, including TNF, IL-1β, IL-6, IL-23 and IL-12 and chemokines, such as CCL5, CCL22, and CCL24, which recruit T cells and other inflammatory cells into the tissue microenvironment. These findings provide solid rationale for antagonizing this signaling pathway in GCA.

The GM-CSF receptor is a member of the haematopoietin receptor superfamily. It is heterodimeric, consisting of an alpha and a beta subunit. The alpha subunit is highly specific for GM-CSF, whereas the beta subunit is shared with other cytokine receptors, including IL-3 and IL-5. This is reflected in a broader tissue distribution of the beta receptor subunit. The alpha subunit, GM-CSFRα, is primarily expressed on myeloid cells and non-haematopoietic cells, such as neutrophils, macrophages, eosinophils, dendritic cells, endothelial cells and respiratory epithelial cells. Full length GM-CSFRα is a 400 amino acid type I membrane glycoprotein that belongs to the type I cytokine receptor family and consists of a 22 amino acid signal peptide (positions 1-22), a 298 amino acid extracellular domain (positions 23-320), a transmembrane domain from positions 321-345 and a short 55 amino acid intra-cellular domain. The signal peptide is cleaved to provide the mature form of GM-CSFRα as a 378 amino acid protein. Complementary DNA (cDNA) clones of the human and murine GM-CSFRα are available and, at the protein level, the receptor subunits have 36% identity. GM-CSF is able to bind with relatively low affinity to the α subunit alone (Kd 1-5 nM) but not at all to the β subunit alone. However, the presence of both α and β subunits results in a high affinity ligand-receptor complex (Kd˜100 pM). GM-CSF signaling occurs through its initial binding to the GM-CSFRα chain and then cross-linking with a larger subunit the common β chain to generate the high affinity interaction, which phosphorylates the JAK-STAT pathway. This interaction is also capable of signaling through tyrosine phosphorylation and activation of the MAP kinase pathway.

Pathologically, GM-CSF has been shown to play a role in exacerbating inflammatory, respiratory and autoimmune diseases. Neutralization of GM-CSF binding to GM-CSFRαis therefore a therapeutic approach to treating diseases and conditions mediated through GM-CSFR. Accordingly, the invention relates to a binding member that inhibits the binding of human GM-CSF to GM-CSFRα, and/or inhibits signaling that results from GM-CSF ligand binding to the receptor, such as, for example, a binding member (e.g. an antibody) that binds human GM-CSF or GM-CSFRα. Upon ligand binding, GM-CSFR triggers stimulation of multiple downstream signaling pathways, including JAK2/STAT5, the MAPK pathway, and the PI3K pathway; all relevant in activation and differentiation of myeloid cells. The binding member may be a reversible inhibitor of GM-CSF signaling through the GM-CSFR.

### Treatment

One aspect of the invention provides methods of treatment for GCA by administering to a subject in need an effective dose of a GM-CSF antagonist (e.g., a GM-CSFRα antagonist), at an effective dose interval, for an effective period of time. In some embodiments, the GM-CSF antagonist a therapeutic anti-GM-CSF monoclonal antibody. Anti-GM-CSF monoclonal antibodies described in international application PCT/EP2006/004696 filed on May 17, 2006, which published as WO2006/122797, and international application PCT/EP2016/076225, which published as WO2017/076804, are hereby incorporated by reference in its entirety. In some embodiments, the GM-CSFRα antagonist is a therapeutic anti-GM-CSFRα monoclonal antibody. Anti-GM-CSFRα monoclonal antibodies described in the international application PCT/GB2007/001108 filed on Mar. 27, 2007 which published as WO2007/110631, the EP application 120770487 filed on Oct. 10, 2010, U.S. application Ser. No. 11/692,008 filed on Mar. 27, 2007, U.S. application Ser. No. 12/294,616 filed on Sep. 25, 2008, U.S. application Ser. No. 13/941,409 filed on Jul. 12, 2013, U.S. application Ser. No. 14/753,792 filed on Nov. 30, 2010, international application PCT/EP2012/070074 filed on Oct. 10, 2012, which published as WO/2013/053767, international application PCT/EP2015/060902 filed on May 18, 2015, which published as WO2015/177097, and international application PCT/EP2017/062479, filed on May 23, 2017, are hereby incorporated by reference in their entirety. In one embodiment, the GM-CSFRα monoclonal antibody is mavrilimumab. WO2007/110631 reports the isolation and characterization of the anti-GM-CSFRα antibody mavrilimumab and variants of it, which share an ability to neutralize the biological activity of GM-CSFRα with high potency. The functional properties of these antibodies are believed to be attributable, at least in part, to binding a Tyr-Leu-Asp-Phe-Gln motif at positions 226 to 230 of human GM-CSFRα, thereby inhibiting the association between GM-CSFRα and its ligand GM-CSF. Mavrilimumab is a human IgG4 monoclonal antibody designed to modulate macrophage activation, differentiation and survival by targeting the GM-CSFRα. It is a potent neutralizer of the biological activity of GM-CSFRα and, was shown to exert therapeutic effects by binding GM-CSFRα on leukocytes within the synovial joints of RA patients, leading to reduced cell survival and activation. The safety profile of the GM-CSFRα antibody mavrilimumab for in vivo use to date has been established in a Phase II clinical trial for rheumatoid arthritis (RA).

GCA patients can be stratified into two categories: patients with new-onset disease, and patients with relapsing disease. In the first category, initial diagnosis of GCA takes place within 6 weeks of the treatment initiation. The diagnosis can be done by Westergren erythrocyte sedimentation rate (ESR), with ESR being >30 mm/hour; or the serum C-Reactive Protein (CRP) level being ≥1 mg/dL. Other symptoms may include cranial symptoms of GCA (new-onset localized headache, scalp or temporal artery tenderness, ischemia-related vision loss, or otherwise unexplained mouth or jaw pain upon mastication, jaw claudication or claudication of the extremities, symptoms of PMR, defined as shoulder and/or hip girdle pain associated with inflammatory morning stiffness. More affirmative diagnosis can be performed by TAB or ultrasound. Additionally, evidence of large-vessel vasculitis by angiography or cross-sectional imaging study such as MRI, CT/CTA or PET-CT of the aorta or other great vessels is noted. The relapsing group is characterized by diagnosis of GCA at a time point longer than 6 weeks (>6 weeks) from the treatment initiation. Patients may be characterized as having no remission since the diagnosis of disease as per clinical expectations (refractory non-remitting). A subset of the relapsing category patients may experience or exhibit no symptoms of GCA at the time of initiation of the treatment (resolution of GCA symptom(s) with CRP <1.0 or ESR <20 mm in the first hour).

In one embodiment, the methods according to the invention include treating subjects having new-onset GCA by administering a therapeutically effective amount of a GM-CSF antagonist, such as, for example, an anti-GM-CSFRα monoclonal antibody (e.g., mavrilimumab), or an anti-GM-CSF monoclonal antibody (e.g, namilumab, otilimab, gimsilumab, lenzilumab or TJM-2). In one embodiment, the methods according to the invention include treating subjects having relapsing GCA by administering a therapeutically effective amount of a GM-CSF antagonist, such as, for example, an anti-GM-CSFRα monoclonal antibody (e.g., mavrilimumab), or an anti-GM-CSF monoclonal antibody (e.g, namilumab, otilimab, gimsilumab, lenzilumab or TJM-2). In one embodiment, the methods according to the invention include treating subjects having refractory GCA by administering a therapeutically effective amount of a GM-CSF antagonist, such as, for example, an anti-GM-CSFRα monoclonal antibody (e.g. mavrilimumab), or an anti-GM-CSF monoclonal antibody (e.g, namilumab, otilimab, gimsilumab, lenzilumab or TJM-2). In one embodiment, the methods according to the invention include treating the subject with an effective dose of mavrilimumab at a dose interval for a treatment period sufficient to improve, stabilize or reduce one or more signs and/or symptoms of GCA relative to a control. The terms, “treat” or “treatment,” as used herein, refers to amelioration of one or more signs and/or symptoms associated with the disease or disorder, prevention or delay of the onset or progression of one or more signs and/or symptoms of the disease or disorder, and/or lessening of the severity or frequency of one or more signs and/or symptoms of the disease or disorder.

In certain embodiments, the subject that is administered a therapeutically effective amount of GM-CSF antagonist (e.g., anti-GM-CSFRα monoclonal antibody or anti-GM-CSF monoclonal antibody) may also be treated concomitantly with other medications, such including immunomodulatory drugs, such as methotrexate or corticosteroids, and combinations thereof, corticosteroids, and combinations thereof, and optionally weaned from one or more of such concomitant medications following treatment with the GM-CSF antagonist (e.g., anti-GM-CSFRα monoclonal antibody or anti-GM-CSF monoclonal antibody). In some embodiments the subject is gradually weaned from a corticosteroid after the GM-CSF antagonist therapy (e.g., anti-GM-CSFRα monoclonal antibody therapy or anti-GM-CSF monoclonal antibody therapy) is initiated. In one embodiment, the corticosteroid is prednisone. In another embodiment, the corticosteroid is methylprednisolone.

GM-CSF antagonist treatment (e.g, anti-GM-CSFRα treatment or anti-GM-CSF treatment) may be given orally (for example nanobodies), by injection (for example, subcutaneously, intravenously, intra-arterially, intra-articularly, intraperitoneal or intramuscularly), by inhalation, by the intravesicular route (instillation into the urinary bladder), or topically (for example intraocular, intranasal, rectal, into wounds, on skin). The treatment may be administered by pulse infusion, particularly with declining doses of the inhibitor. The route of administration can be determined by the physicochemical characteristics of the treatment, by special considerations for the disease or by a requirement to optimize efficacy or to minimize side-effects. In some embodiments, subcutaneous injection of the GM-CSF antagonist (e.g., the anti-GM-CSFRα monoclonal antibody or anti-GM-CSF monoclonal antibody) can be performed in the upper arm, the anterior surface of the thigh, the lower portion of the abdomen, the upper back or the upper area of the buttock. In some embodiments, the site of injection is rotated.

In certain embodiments, the treatment results in a reduction or elimination of symptoms associated with GCA. In some embodiments, the treatment reduces arterial inflammation and/or reduces expression of genes associated with GCA lesions. Accordingly, in certain embodiments, treatment results in the reduction of protein and/or RNA expression of one or more of GM-CSF, GM-CSFRα, JAK2, IL-6, CD83, PU.1, HLA-DRA, CD3E, TNFα, IL-1β, or combinations thereof. In some embodiments, treatment results in the reduction or elimination of infiltrated macrophages. In another embodiment, the treatment reduces T-cells in the vessel adventitia. In one embodiment, treatment results in a reduction of GM-CSFRα expression in vasa vasorum of the temporal artery. In some embodiments, the density of the inflammatory infiltrates is suppressed and/or the vessel wall remodeling (e.g., intimal hyperplasia, luminal stenosis and tissue ischemia) is regressed, improved, stabilized or reduced. In one embodiment, treatment results in a reduction of cells positive for GM-CSF or INF-γ in the arterial wall. In other embodiments, the treatment normalizes gene expression levels, or improves gene expression levels (i.e., expression levels that are between a subject with GCA and a subject who does not have GCA), of one or more genes related to interferon signaling, IL-6 signaling or GM-CSF signaling. Genes related to interferon signaling include, without limitation, INF-γ, INF-αR1, INF-γR1, INF-γR2, IFI30, IFI35, PRKCD, B2M, IFNAR1, CIITA, PTPN2, PTPN11, IRF1, IFR5, IRF8, GBP1, GBP5, STAT1, STAT2, FCγR1A/B, ICAM1, VCAM1, TYK2, CD44, IP6K2, DDX58 and PTPN6. Genes related to IL-6 signaling include, without limitation, PTPN11, TYK2, STAT1, IL-11RA and IL-6. Genes related to GM-CSF signaling include, without limitation, IL-2RB, IL-2RG, GM-CSFRα, JAK3, STAT5A, SYK, PTPN11, HCK, FYN, INPP5D, BLNK and PTPN6.

Dosage

A therapeutically effective dose of a GM-CSF antagonist (e.g., anti-GM-CSFRα antibody or anti-GM-CSF monoclonal antibody) for treating GCA can occur at various dosages. In some embodiments of the invention, a therapeutically effective dose is equal to or greater than 0.1 mg/kg, 0.3 mg/kg, 0.5 mg/kg, 0.7 mg/kg, 1 mg/kg. 1.25 mg/kg, 1.5 mg/kg, 1.75 mg/kg, 2 mg/kg, 2.5 mg/kg, 3.5 mg/kg, 4 mg/kg, or 5 mg/kg, or 10 mg/kg.

In some embodiments, a therapeutically effective dose is approximately 0.1-10 mg/kg, approximately 0.2-10 mg/kg, approximately 0.3-10 mg/kg, approximately 0.4-10 mg/kg, approximately 0.5-10 mg/kg, approximately 0.6-10 mg/kg, approximately 0.7-10 mg/kg, approximately 0.8-10 mg/kg, approximately 0.9-10 mg/kg, approximately 1-10 mg/kg, approximately 2-10 mg/kg, approximately 3-10 mg/kg, approximately 5-10 mg/kg, or any range in between. In some embodiments, the approximately 0.3-5 mg/kg, or approximately 0.3-4 mg/kg or approximately 0.3-3 mg/kg. In some embodiments, the therapeutically effective dose is approximately 0.5-2.5 mg/kg.

In some embodiments, administering comprises an initial bolus or loading dose, followed by at least one maintenance dose. In some embodiments, the initial bolus or loading dose is greater than the at least one maintenance dose. In some embodiments, the initial bolus or loading dose is at least onefold, twofold, threefold, fourfold or fivefold greater in dosage than the dosage of the at least one maintenance dose. In some embodiments, the initial bolus or loading dose is twofold greater in dosage than the dosage of the at least one maintenance dose.

In some embodiments, a fixed dose is used as an initial dose and/or maintenance dose. A suitable fixed dose may be equal to or greater than about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 210 mg, about 220 mg, about 225 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg or about 400 mg. In certain embodiments, fixed dose used as an initial dose and/or maintenance dose is 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 210 mg, 220 mg, 225 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg or 400 mg. In some embodiments, a suitable fixed dose ranges from 50-500 mg, 100-400 mg, 150-400 mg, 200-400 mg, 250-400 mg, 300-350 mg, 320-400 mg, or 350-400 mg. In some embodiments, a suitable fixed dose is 150 mg. In some embodiments, a suitable fixed dose is provided in a single injection syringe. A suitable fixed dose may be administered (e.g., subcutaneously or intravenously) in a single injection or by multiple injections.

In some embodiments the treatment with the effective dose of GM-CSF antagonist (e.g., anti-GM-CSFRα antibody or anti-GM-CSF monoclonal antibody) is accompanied by corticosteroid treatment. The patient may be on corticosteroid prior to treatment with GM-CSF antagonist therapy (e.g., anti-GM-CSFRα antibody therapy or anti-GM-CSF monoclonal antibody therapy). The concomitant steroid dose may comprise about 25 mg, or about 30 mg, or about 40 mg, or about 50 mg, or about 60 mg, or about 70 mg, or about 80 mg, or about 100 mg, or about 110 mg, or about 120 mg, or about 125 mg of prednisone. In some embodiments, the concomitant dose is 25 mg, or 30 mg, or 40 mg, or 50 mg, or 60 mg, or 70 mg, or 80 mg, or 100 mg, or 110 mg, or 120 mg, or 125 mg of prednisone.

Administration Interval

An administration interval of a GM-CSF antagonist (e.g., an anti-GM-CSFRα antibody or anti-GM-CSF monoclonal antibody) in the treatment of GCA can occur at various durations. In some embodiments of the invention, the administration interval is daily. In some embodiments, the administration interval is every other day. In some embodiments, the administration interval is multiple times a week. In some embodiments, the administration interval is once every week. In some embodiments, the administration interval is once every two weeks. In some embodiments, the administration interval is once every three weeks. In some embodiments, the administration interval is once every four weeks. In some embodiments, the administration interval is once every five weeks.

Treatment Period

A treatment period of GCA with a GM-CSF antagonist (e.g., an anti-GM-CSFRα antibody or anti-GM-CSF monoclonal antibody) can vary in duration. In some embodiments, the treatment period is at least one month. In some embodiments, the treatment period is at least two months. In some embodiments, the treatment period is at least three months. In some embodiments, the treatment period is at least six months. In some embodiments, the treatment period is at least nine months. In some embodiments, the treatment period is at least one year. In some embodiments, the treatment period is about 20 weeks. In some embodiments, the treatment period is about 21 weeks, or about 22 weeks or about 23 weeks or about 24 weeks or about 25 weeks, or about 26 weeks, or about 27 weeks, about 28 weeks, or about 29 weeks, or about 30 weeks, or about 31 weeks, or about 32 weeks, or about 33 weeks or about 34 weeks or about 35 weeks, or about 36 weeks, or about 37 weeks, or about 38 weeks, or about 39 weeks, or about 40 weeks, or about 41 weeks, or about 42 weeks, or about 43 weeks or about 44 weeks or about 45 weeks, or about 46 weeks, or about 47 weeks, or about 48 weeks, or about 49 weeks or about 50 weeks, or about 51 weeks, or about 52 weeks. In some embodiments, the treatment period is about 26 weeks. In one embodiment, the treatment period is 26 weeks. In one embodiment, the treatment period is 52 weeks In some embodiments, the treatment period is 21 weeks, or 22 weeks, or 23 weeks, or 24 weeks, or 25 weeks, 26 weeks, 27 weeks, 28 weeks, 29 weeks or 30 weeks, or 31 weeks, or 32 weeks, or 33 weeks, or 34 weeks, or 35 weeks, 36 weeks, 37 weeks, 38 weeks, 39 weeks, or 40 weeks, or 41 weeks, or 42 weeks or 43 weeks or 44 weeks or 45 weeks, 46 weeks, 47 weeks, 48 weeks, 49 weeks or 50 weeks, or 51 weeks, or 52 weeks. In one embodiment, the treatment period is 26 weeks. In one embodiment, the treatment period is 52 weeks. In some embodiments, the treatment period is at least two years. In some embodiments, the treatment period continues throughout the subject's life.

Pharmacokinetics and Pharmacodynamics

Evaluation of anti-GM-CSFRα antibody concentration-time profiles in serum of subjects with atopic dermatitis may be evaluated directly by measuring systemic serum anti-GM-CSFRα antibody concentration-time profiles. Typically, anti-GM-CSFRα antibody pharmacokinetic and pharmacodynamic profiles are evaluated by sampling the blood of treated subjects periodically. The following standard abbreviations are used to represent the associated pharmacokinetic parameters.
Cₘₐₓ maximum concentration
tₘₐₓ time to maximum concentration
   AUC₀₋ₜ area under the concentration-time curve (AUC) from time zero to the last measurable concentration, calculated using the linear trapezoidal rule for increasing concentrations and the logarithmic rule for decreasing concentrations
AUC_{0-∞} AUC from time zero to infinity, calculated using the formula:

$${AUC}_{0 - ∞} = {AUC}_{0 - t} + \frac{{C}_{t}}{{λ}_{z}}$$

where Cₜ is the last measurable concentration and λ_{Z} is the apparent terminal elimination rate constant
λ_{Z} apparent terminal elimination rate constant, where λ_{Z} is the magnitude of the slope of the linear regression of the log concentration versus time profile during the terminal phase
t_{1/2} apparent terminal elimination half-life (whenever possible), where

t_{1/2}=natural log(ln)(2)/λ_{Z}
CL clearance
Vd volume of distribution (IV doses only)
Vd/F apparent volume of distribution (SC doses only)

Typically, actual blood sample collection times relative to the start of anti-GM-CSFRα antibody administration are used in PK analysis. For example, blood samples are typically collected within 15 or 30 minutes prior to anti-GM-CSFRα antibody administration (pre-administration baseline or time 0) and at hours 1, 4, 8 or 12, or days 1 (24 hours), 2, 3, 4, 5, 6, 7, 10, 14, 17, 21, 24, 28, 31, 38, 45, 52, 60, 70 or 90 days, following administration.

Various methods may be used to measure anti-GM-CSFRα antibody concentration in serum. As a non-limiting example, enzyme-linked immunosorbent assay (ELISA) methods are used.

Pharmacokinetic parameters may be evaluated at any stage during the treatment, for example, at day 1, day 2, day 3, day 4, day 5, day 6, week 1, week 2, week 3, week 4, week 5, week 6, week 7, week 8, week 9, week 10, week 11, week 12, week 13, week 14, week 15, week 16, week 17, week 18, week 19, week 20, week 21, week 22, week 23, week 24, or later. In some embodiments, pharmacokinetic parameters may be evaluated at month 1, month 2, month 3, month 4, month 5, month 6, month 7, month 8, month 9, month 10, month 11, month 12, month 13, month 14, month 15, month 16, month 17, month 18, month 19, month 20, month 21, month 22, month 23, month 24, or later during the treatment.

Adverse Effects

Mavrilimumab (CAM-3001) completed phase II clinical trials for rheumatoid arthritis (RA) with long term safety studies performed, which are reported in international applications PCT/EP2012/070074 filed on Oct. 10, 2012 (WO 2013/053767), and PCT/EP2015/060902 (WO2015177097) both are incorporated herein by reference. In both the cases the drug was well tolerated.

In some embodiments, administration of a GM-CSF antagonist (e.g., an anti-GM-CSFRα antibody or anti-GM-CSF antibody) at a dose of up to 150 mg for an extension of up to about 150 weeks results in no serious adverse effects in the subjects. In some embodiments, administration of a GM-CSF antagonist (e.g., an anti-GM-CSFRα antibody or anti-GM-CSF antibody) at a dose of up to 150 mg for 52 weeks results in no serious infections, or no serious infections. In some embodiments, administration of a GM-CSF antagonist (e.g., an anti-GM-CSFRα antibody or anti-GM-CSF monoclonal antibody) does not result in adverse pulmonary function or hematological function. Based on data from the clinical trial, a similar percentage of pulmonary AEs occurred on the active and placebo groups. There were no cases of pulmonary alveolar proteinosis (PAP) or suggestive of PAP. Two pneumonia cases were reported: (i) in the placebo group, presenting non-serious infection with pleural effusion and (ii) in the 30 mg dose group, presenting serious infection. There were no other serious infections. There was one case where ALT >3X ULN and Bili >2X ULN due to cholelithiasis but no other clinically meaningful laboratory abnormalities. No anaphylaxis reaction was reported. Two hypersensitivity AEs leading to discontinuation (drug hypersensitivity on 30 mg and angioedema on 150 mg) were observed.

### GM-CSF Antagonists

Any GM-CSF antagonists can be used to practice the present invention. GM-CSF antagonist may function by blocking GM-CSF from interaction with the GM-CSF receptor alpha or the GM-CSF receptor beta, or by blocking formation of heterodimers of these proteins, and as such prevent GM-CSF binding and/or signaling thereby reducing production of cytokines and/or activation of monocytes and macrophages. The GM-CSF antagonists according to the invention may therefore be a binding agent (e.g., an antibody or compound) of either GM-CSF or one or more of the GM-CSFR receptors (i.e., GM-CSFRα or GM-CSFRβ) or an agent capable of interfering with these interactions in a manner which affects GM-CSF biological activity. Herein, reference to a GM-CSF antagonist can be taken to mean either an antagonist to GM-CSF or to one of its receptors.

Anti-GM-CSF Antibodies

In some embodiments, inventive compositions and methods provided by the present invention are used to deliver an anti-GM-CSF antibody or fragment thereof to a subject in need. The anti-GM-CSF antibodies administered these methods may be an IgG subclass antibody, in some embodiments an IgG1, IgG2 or IgG4 subclass antibody. The anti-GM-CSF antibody may be a monoclonal antibody. In certain embodiments of the invention, the anti-GM-CSF antibody is namilumab. In some embodiments, the anti-GM-CSF antibody is otilimab. In some embodiments, the anti-GM-CSF antibody is gimsilumab. In some embodiments, the anti-GM-CSF antibody is lenzilumab. In some embodiments, the anti-GM-CSF antibody is TJM-2.

Anti-GM-CSF Receptor Alpha (GM-CSFRα) Antibodies

In some embodiments, inventive compositions and methods provided by the present invention are used to deliver an anti-GM-CSFRα antibody to a subject in need. In certain embodiments of the invention, the anti-GM-CSFRα antibody is mavrilimumab. The isolation and characterization of mavrilimumab is described in WO2007/110631 and WO2013/053767, both are fully incorporated by reference in their entireties. Mavrilimumab is human IgG4 monoclonal antibody that specifically inhibits GM-CSFRα mediated signaling, that is, GM-CSF activated cell signaling. In certain embodiments, the antibody is comprised of two light chains and two heavy chains. The heavy chain variable domain (VH) comprises an amino acid sequence identified in SEQ ID NO: 1. The light chain variable domain (VL) comprises an amino acid sequence identified in SEQ ID NO: 2. The heavy and light chains each comprise complementarity determining regions (CDRs) and framework regions in the following arrangement:
FR1-CDR1-FR2-CDR2-FR3-CDR3-FR4

The mavrilimumab antibody heavy chain comprises CDRs: HCDR1, HCDR2, HCDR3 as identified by the amino acid sequences in SEQ ID NO: 3, 4 and 5 respectively. The light chain comprises CDRs: LCDR1, LCDR2, LCDR3 as identified by the amino acid sequences in SEQ ID NO: 6, 7 and 8 respectively.

Anti-GM-CSFRα Heavy Chain Variable Domain Amino
Acid Sequence
(SEQ ID NO: 1)
QVQLVQSGAEVKKPGASVKVSCKVSGYTLTELSIHWVRQAPGKGLEW
MGGFDPEENEIVYAQRFQGRVTMTEDTSTDTAYMELSSLRSEDTAVY
YCAIVGSFSPLTLGLWGQGTMVTVSS
Anti-GM-CSFRα Light Chain Variable Domain Amino
Acid Sequence
(SEQ ID NO: 2)
QSVLTQPPSVSGAPGQRVTISCTGSGSNIGAPYDVSWYQQLPGTAPK
LLIYHNNKRPSGVPDRFSGSKSGTSASLAITGLQAEDEADYYCATVE
AGLSGSVFGGGTKLTVL
Anti-GM-CSFRa Heavy Chain Variable Domain CDR1
(HCDR1) Amino Acid Sequence
(SEQ ID NO: 3)
ELSIH
Anti-GM-CSFRα Heavy Chain Variable Domain CDR2
(HCDR2) Amino Acid Sequence
(SEQ ID NO: 4)
GFDPEENEIVYAQRFQG
Anti-GM-CSFRα Heavy Chain Variable Domain CDR3
(HCDR3) Amino Acid Sequence
(SEQ ID NO: 5)
VGSFSPLTLGL
Anti-GM-CSFRα Light Chain Variable Domain CDR1
(LCDR1) Amino Acid Sequence
(SEQ ID NO: 6)
TGSGSNIGAPYDVS
Anti-GM-CSFRα Light Chain Variable Domain CDR2
(LCDR2) Amino Acid Sequence
(SEQ ID NO: 7)
HNNKRPS
Anti-GM-CSFRa Light Chain Variable Domain CDR3
(LCDR3) Amino Acid Sequence
(SEQ ID NO: 8)
ATVEAGLSGSV

In some embodiments the anti-GM-CSFRα antibody for GCA treatment is a variant of mavrilimumab, selected from the GM-CSFα binding members disclosed in the application WO2007/11063 and WO2013053767, which is incorporated by reference in its entirety.

In some embodiments the anti-GM-CSFRα antibody for GCA treatment comprises CDR amino acid sequences with at least 75%, 80%, 85%, 90%, 91%, 92%, 93%, 94%, 95%, 96%, 97%, 98%, 99% or more identity with one or more of SEQ ID NO:3, SEQ ID NO: 4, SEQ ID NO: 5, SEQ ID NO: 6, SEQ ID NO: 7, and SEQ ID NO: 8.

In some embodiments the anti-GM-CSFRα antibody comprises a light chain variable domain having an amino acid sequence at least 90% identical to SEQ ID NO: 2 and a heavy chain variable domain having an amino acid sequence at least 90% identical to SEQ ID NO: 1. In some embodiments of the invention, an anti-GM-CSFRα antibody has a light chain variable domain amino acid sequence with at least 50%, 55%, 60%, 65%, 70%, 75%, 80%, 85%, 90%, 91%, 92%, 93%, 94%, 95%, 96%, 97%, 98%, 99% or more identity to SEQ ID NO: 2 and a heavy chain variable domain amino acid sequence with at least 50%, 55%, 60%, 65%, 70%, 75%, 80%, 85%, 90%, 91%, 92%, 93%, 94%, 95%, 96%, 97%, 98%, 99% or more identity to SEQ ID NO: 1. In some embodiments of the invention, an anti-GM-CSFRα antibody comprises a light chain variable domain that has the amino acid sequence set forth in SEQ ID NO: 2 and a heavy chain variable domain that has the amino acid sequence set forth in SEQ ID NO: 1. In some embodiments of the invention, a heavy chain constant region of an anti-GM-CSFRα antibody comprises CH1, hinge and CH2 domains derived from an IgG4 antibody fused to a CH3 domain derived from an IgG1 antibody. In some embodiments of the invention, a heavy chain constant region of an anti-GM-CSFRα antibody is, or is derived from, an IgG1, IgG2 or IgG4 heavy chain constant region. In some embodiments of the invention, a light chain constant region of an anti-GM-CSFRα antibody is, or is derived from, a lambda or kappa light chain constant region.

In some embodiments, the anti-GM-CSFRα inhibitor is a fragment of mavrilimumab antibody. In some embodiments the inhibitor comprises a single chain variable fragment (ScFv) comprising at least any one of the CDR sequences of SEQ ID NO: 3, 4, 5, 6, 7, or 8. In some embodiments the inhibitor is a fusion molecule comprising at least any one of the CDR sequences of SEQ ID NO: 3, 4, 5, 6, 7, or 8. In some embodiments, the anti-GM-CSFRα inhibitor sequence is a bispecific antibody comprising at least one of the CDR sequences of SEQ ID NO: 3, 4, 5, 6, 7, or 8.

Pharmaceutical Composition

In one aspect, the invention provides a pharmaceutical composition comprising an anti-GM-CSF antibody (e.g., anti-GM-CSFRα antibody) is a liquid product intended for SC administration. In some embodiments, is the pharmaceutical composition stored at 2° C. to 8° C. (36° C. to 46° F.). In one embodiment, the drug is formulated at 150 mg/mL in 50 mM sodium acetate, 70 mM sodium chloride, 4% (weight/volume [w/v]) trehalose dihydrate, 0.05% (w/v) polysorbate 80, pH 5.8. In some embodiments, the pharmaceutical product is supplied as a sterile liquid, in a prefilled syringe at a nominal fill volume of 1.0 mL, stoppered with a Teflon-faced elastomeric stopper, and accessorized with a needle guard, plunger rod and extended finger flange. Each syringe contains 150 mg (nominal) of active investigational product.

### EXAMPLES

While certain methods of the present invention have been described with specificity in accordance with certain embodiments, the following examples serve only to illustrate the methods of the invention and are not intended to limit the same.

### Example 1: Treatment of Giant Cell Arteritis with Anti-GM-CSFRα Antibody

The study in this example is designed to evaluate the efficacy of an anti-GM-CSFRα antibody in treating subjects with GCA.

### Study Design

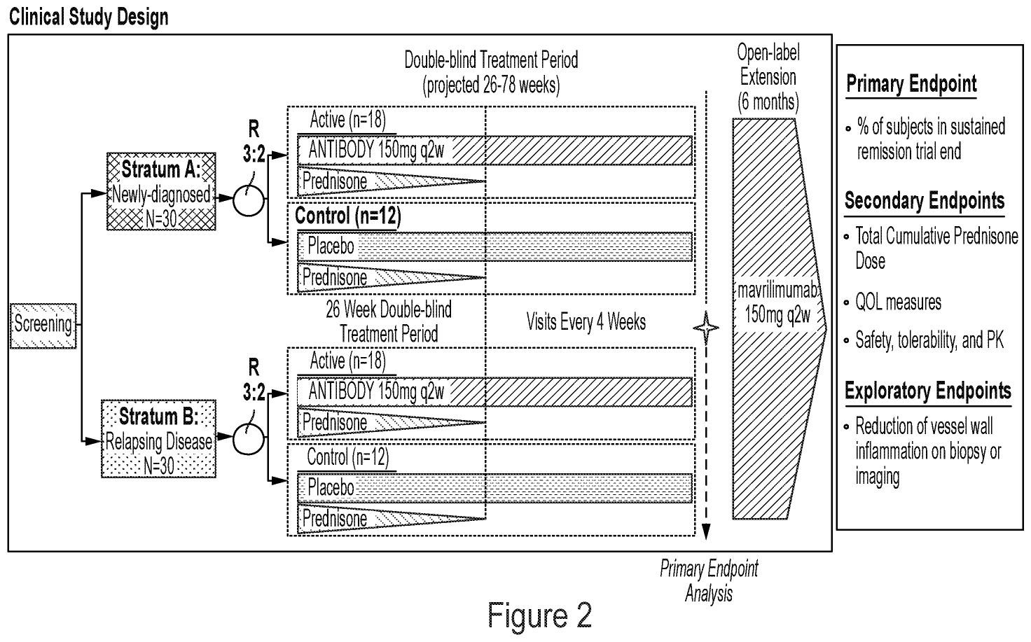

In this exemplary randomized, double-blinded, placebo-controlled study design, anti-GM-CSFRα antibody (mavrilimumab) is co-administered with a 26 week steroid taper to subjects who are clinically diagnosed with GCA (early onset and relapsing/refractory) in order to evaluate efficacy and safety of mavrilimumab. The study design is exemplified in FIG. 2. The study consists of a screening period (up to 6 weeks), a double-blind placebo-controlled period during which subjects will receive blinded mavrilimumab or placebo, a 26-week corticosteroid taper until the last subject has reached the 26-week time point and the results from the 26-week time point have been analyzed, and an Open-Label Extension (OLE) for an additional 26-week period.

The exploratory objectives of the study include evaluating the reduction of vessel wall inflammation on biopsy (in consenting subjects) or imaging at Week 26 compared to baseline, and evaluating the association between blood pharmacodynamic (PD) biomarkers and assessments of clinical response. Ultrasound tests are performed at Weeks 12 and 26, and every 6 months.

Subjects are permitted to have received steroids (prednisone or equivalent) prior to inclusion in the study. Subjects receive concomitant medications in line with current standard of care (SoC) practices for GCA. Such medications include low-dose aspirin (dose allowed per SoC), pantoprazole (40 mg daily), calcium (1000 mg daily), cholecalciferol (800 U daily), and intravenous (IV) ibandronate 3 mg every 3 months, for the duration of the study.

Subjects receive subcutaneous (SC) mavrilimumab or placebo as well as co-administered oral prednisone, which is tapered over a period of up to 26 weeks, unless the subject experiences a flare of GCA. Upon flare the subject remains on blinded therapy and the dose of the steroid is increased, or optionally, upon flare, the subject is discontinued from study drug, SoC is administered, and the subject is followed for the remainder of the study.

Subjects receive mavrilimumab or placebo for a minimum of 26 weeks (unless a subject discontinues treatment prematurely). All subjects are offered an open-label mavrilimumab extension for an additional 6 months.

Safety measures include adverse events and clinical laboratory analyses (including chemistry, hematology, urinalysis, liver profiles, lipid panel, hemoglobin A1c [HbA1c], and anti-drug antibodies), vital sign measurements, electrocardiograms (ECGs), and physical examination findings.

### Drug Formulations

### Mavrilimumab

Mavrilimumab is a liquid product intended for SC administration. It must be stored at 2° C. to 8° C. (36° C. to 46° F.). Mavrilimumab is formulated at 150 mg/mL in 50 mM sodium acetate, 70 mM sodium chloride, 4% trehalose dihydrate, 0.05% (weight/volume [w/v]) polysorbate 80, pH 5.8. The investigational product is supplied as a sterile liquid, in a prefilled syringe at a nominal fill volume of 1.0 mL, stoppered with a Teflon-faced elastomeric stopper, and accessorized with a needle guard, plunger rod and extended finger flange. Each syringe contains 150 mg (nominal) of active investigational product.

### Placebo

Mavrilimumab placebo is a liquid product intended for SC administration. It must be stored at 2° C. to 8° C. (36° C. to 46° F.). Mavrilimumab placebo is formulated in 50 mM sodium acetate, 70 mM sodium chloride, 4% trehalose dihydrate, 0.05% (w/v) polysorbate 80, pH 5.8. The placebo is supplied as a sterile liquid, in a prefilled syringe at a nominal fill volume of 1.0 mL, stoppered with a Teflon-faced elastomeric stopper, and accessorized with a needle guard, plunger rod and extended finger flange.

### Prednisone

Prednisone Tablets USP are available for oral administration containing either 1 mg, or 2.5 mg, 5 mg, 10 mg, 20 mg or 50 mg of prednisone USP. Each tablet contains the following inactive ingredients: lactose monohydrate, magnesium stearate, microcrystalline cellulose, pregelatinized starch, sodium starch glycolate, and stearic acid (1 mg, 2.5 mg, and 5 mg only).

### Study Treatments

During the double-blind period, subjects receive blinded mavrilimumab 150 mg or placebo, every 2 weeks, by SC injection, in addition to a protocol-specific corticosteroid taper.

Oral prednisone is started at a dose between 20 mg/day to 60 mg/day (inclusive) at Day 0 depending on the subject's previous steroid treatment, disease status, and Investigator discretion. The prednisone dose is then tapered over the subsequent 26 weeks in accordance with the following tapering schedule shown in Table 1 (in absence of a GCA flare), with subjects entering the taper at different points, depending on their prednisone dose at Day 0.

Duration of treatment can differ according to when each subject is enrolled, with the first enrolling subjects receiving treatment for longer than those who enroll later. By the time all subjects have completed 26 weeks of treatment and the 26-week results have been analyzed, some subjects (those who enroll early in the recruitment process) will have received blinded mavrilimumab or placebo for approximately 21 months. Depending on the results from the 26-week analysis, all subjects are offered open-label mavrilimumab for an additional 6 months. Thus, the approximate total duration of treatment will be up to 27 months.

TABLE 1
Prednisone Tapering Schedule

| Prednisone | | | | | | | |
|---|---|---|---|---|---|---|---|
| Dose | Prednisone Dose at Study Start (Day 0) | | | | | | |
| (mg/day) | 60 mg | 50 mg | 40 mg | 35 mg | 30 mg | 25 mg | 20 mg |
| 60 | Week 1 | | | | | | |
| 50 | Week 2 | Week 1 | | | | | |
| 40 | Week 3 | Week 2 | Week 1 | | | | |
| 35 | Week 4 | Week 3 | Week 2 | Week 1 | | | |
| 30 | Week 5 | Week 4 | Week 3 | Week 2 | Week 1 | | |
| 25 | Week 6 | Week 5 | Week 4 | Week 3 | Week 2 | Week 1 | |
| 20 | Week 7 | Week 6 | Week 5 | Week 4 | Week 3 | Week 2-3 | Week 1-3 |
| 17.5 | Week 8 | Week 7 | Week 6 | Week 5 | Week 4 | Week 4 | Week 4 |
| 15 | Week 9 | Week 8 | Week 7 | Week 6 | Week 5 | Week 5 | Week 5 |
| 12.5 | Week 10 | Week 9 | Week 8 | Week 7 | Week 6 | Week 6 | Week 6 |
| 12.5 | Week 11 | Week 10 | Week 9 | Week 8 | Week 7 | Week 7 | Week 7 |
| 10 | Week 12 | Week 11 | Week 10 | Week 9 | Week 8 | Week 8 | Week 8 |
| 9 | Week 13 | Week 12 | Week 11 | Week 10 | Week 9 | Week 9 | Week 9 |
| 8 | Week 14 | Week 13 | Week 12 | Week 11 | Week 10 | Week 10 | Week 10 |
| 7 | Week 15 | Week 14 | Week 13 | Week 12 | Week 11 | Week 11 | Week 11 |
| 6 | Week 16 | Week 15 | Week 14 | Week 13 | Week 12 | Week 12 | Week 12 |
| 5 | Week 17 | Week 16 | Week 15 | Week 14 | Week 13 | Week 13 | Week 13 |
| 5 | Week 18 | Week 17 | Week 16 | Week 15 | Week 14 | Week 14 | Week 14 |
| 4 | Week 19 | Week 18 | Week 17 | Week 16 | Week 15 | Week 15 | Week 15 |
| 4 | Week 20 | Week 19 | Week 18 | Week 17 | Week 16 | Week 16 | Week 16 |
| 3 | Week 21 | Week 20 | Week 19 | Week 18 | Week 17 | Week 17 | Week 17 |
| 3 | Week 22 | Week 21 | Week 20 | Week 19 | Week 18 | Week 18 | Week 18 |
| 2 | Week 23 | Week 22 | Week 21 | Week 20 | Week 19 | Week 19 | Week 19 |
| 2 | Week 24 | Week 23 | Week 22 | Week 21 | Week 20 | Week 20 | Week 20 |
| 1 | Week 25 | Week 24 | Week 23 | Week 22 | Week 21 | Week 21 | Week 21 |
| 1 | Week 26 | Week 25 | Week 24 | Week 23 | Week 22 | Week 22 | Week 22 |

### Subject Inclusion Criteria

Age of subjects is between 50 and 85 years, both inclusive, who are able to provide written informed consent.

New onset GCA patient subset is categorized as having diagnosed within 6 weeks of Day 0 of the study commencement and the active disease state is characterized with:

(a) Westergren method of erythrocyte sedimentation rate (ESR) greater than 30 mm/hour, or blood CRP ≥levels 1 mg/dL along with:
(b) at least one of the following:
i) Unequivocal cranial symptoms of GCA (new-onset localized headache, scalp or temporal artery tenderness, ischemia-related vision loss, or otherwise unexplained mouth or jaw pain upon mastication)
ii) Unequivocal extracranial symptoms of GCA such as claudication of the extremities
iii) Symptoms of PMR, defined as shoulder and/or hip girdle pain associated with inflammatory morning stiffness;
   (c) AND at least one of the following:
i. TAB or ultrasound revealing features of GCA
ii. Evidence of large-vessel vasculitis by angiography or cross-sectional imaging study such as MRI, CT/CTA or PET-CT of the aorta or other great vessels

Relapsing GCA patient subset is categorized as having diagnosed longer than 6 weeks prior to Day 0 of the study commencement and is characterized by

1. a) Clinical signs and symptoms, Westergren ESR >30 mm/hour or CRP >1 mg/dL;
OR
b) No remission since the diagnosis of disease as per clinical expectations (refractory non-remitting)
   2. Remission of GCA at Day 0 (resolution of GCA symptom(s) and CRP <1.0 or ESR <20 mm in the first hour), such that the subject can safely participate in the study and follow the protocol defined procedures, including initiation of the prednisone taper at the protocol-specified starting dose (i.e., ≤60 mg/day).
   3. At screening, subjects receiving or able to receive oral prednisone up to 60 mg/day for the treatment of active GCA.
   4. Where subjects are using methotrexate, oral or parenteral methotrexate up to 25 mg/week is permitted if started more than 6 weeks prior to Day 0 and should be stable or decreasing with the intention to discontinue use once the patient has achieved remission.
   5. Subjects are willing to receive antiplatelet therapy depending on the Investigator's decision.
   6. Subjects are willing to receive treatment for prevention of corticosteroid-induced osteopenia/osteoporosis depending on the Investigator's decision.

Female subjects are postmenopausal, defined as at least 12 months post cessation of menses (without an alternative medical cause), or permanently sterile following documented hysterectomy, bilateral salpingectomy, bilateral oophorectomy, or tubal ligation or having a male partner with vasectomy as affirmed by the subject, or nonpregnant, nonlactating, and having agreed to use an effective method of contraception (i.e., hormonal contraceptives, IUD or double barrier methods such as condom plus diaphragm or diaphragm plus spermicide or condom plus spermicide) from Screening visit until 12 weeks after final study drug administration.

Male subjects must have documented vasectomy or must agree to use double barrier methods of contraception (such as condom plus diaphragm or diaphragm plus spermicide or condom plus spermicide) or use condom plus hormonal contraceptives or condom plus IUD with their partners of childbearing potential from Day 0 until the Safety Follow-up visit. Male agrees to refrain from donating sperm from Day 0 until the Safety Follow-up visit.

### Study Assessments

Blood samples are collected by venipuncture or cannulation, and serum concentrations of the anti-GM-CSFRα antibody are determined using a validated analytical procedure. All statistical analyses are performed using SAS® Version 9.4 or higher. All clinical study data will be presented in subject data listings. Descriptive statistics include number of subjects (n), mean, standard deviation (SD), first quartile (Q1), median, third quartile (Q3), minimum and maximum for continuous variables, and frequency and percentage for categorical and ordinal variables. Descriptive statistics (arithmetic mean, standard deviation, minimum, median, maximum, geometric mean, and geometric coefficient of variation, as appropriate) are listed and summarized for serum concentrations of anti-GM-CSFRα antibody and PK parameters.

The anti-GM-CSFRα antibody dose proportionality is examined between the dose groups. The AUC_{0-∞}, AUC₀₋ₜ, and Cₘₐₓ estimates are tested for dose proportionality using a power model approach or analysis of variance (ANOVA) model as appropriate.

The following clinical response assessments are also conducted during the study. Efficacy Measures were conducted by:
Clinical laboratory analyses (e.g., CRP, ESR)
Clinical GCA assessment, including, for example, 11-point pain Numerical Rating Scale (NRS), and Functional Assessment of Chronic Illness Therapy (FACIT [fatigue])
Imaging studies (as applicable), including ultrasound, MRI, CT/CTA, PET-CT, TAB (if applicable)
Quality of life (QoL) questionnaires (e.g., EQ-5D, Short Form Health Survey [SF-36])

The primary endpoint analysis of the study is to evaluate the efficacy of mavrilimumab versus placebo, in combination with a 26-week steroid taper, for maintaining sustained remission for 26 weeks in subjects with new-onset or relapsing/refractory GCA. Sustained remission is defined as the absence of flare (as defined above) from the start of double-blind treatment through Week 26 and after. The primary endpoint is duration of remission within the 26-week double-blind base period (time from start of double-blind treatment until the first flare occurring within the 26-week period). Subjects who do not experience a flare during that period are censored at the Week 26 visit. Subjects who drop out or who are lost to follow-up prior to experiencing a flare during the 26-week double-blind period are censored at the time of their last available visit. The number and percentage of subjects who remain in remission, who flare, and who are lost to follow-up prior to a flare during the 26-week double-blind period are summarized for each treatment group. Duration of remission is summarized by the 25^{th}, 50^{th} (median), and 75^{th} percentiles calculated using the Kaplan-Meier method to estimate the survival functions for each treatment group. The 95% confidence interval (CI) for the percentiles will also be calculated. A log-rank test is used to compare mavrilimumab and placebo with respect to the duration of remission (test the equality of the survival remission curves). Kaplan-Meier estimates of remission at 26 weeks are presented with the corresponding 95% CI by treatment group. To describe the magnitude of treatment effect, the hazard ratio for mavrilimumab compared to placebo and the corresponding 95% CI is calculated based on a Cox proportional-hazards model with treatment and randomization stratum as covariates. The primary analysis of sustained remission is performed for the mITT population and will be repeated for the PP population as a sensitivity analysis.

As a secondary efficacy endpoint, duration of remission during the entire double-blind treatment period is analyzed using the same methods described above. Subjects who do not experience flare during double-blind treatment are censored at their last visit of the double-blind treatment period. Subjects who drop out or are lost to follow-up prior to experiencing a flare at any time during double-blind treatment will be censored at the time of their last available visit. The secondary objectives of the study, in subjects with new-onset and relapsing/refractory GCA, are:
To evaluate the effect of mavrilimumab vs placebo on cumulative corticosteroid dose.
To evaluate the effect of mavrilimumab vs placebo on health-related quality of life (HRQoL).
To evaluate the safety and tolerability of mavrilimumab.
To evaluate the pharmacokinetics (PK) of mavrilimumab.
The Hospital Anxiety and Depression Scale (HADS) is a general Likert scale used to detect states of anxiety and depression. The 14 items on the questionnaire include 7 that are related to anxiety and 7 that are related to depression. Each item on the questionnaire is scored on a scale of 0 to 3 with a possible total score between 0 and 21 for each parameter.

Additional secondary efficacy endpoints include the following dichotomous endpoints that are analyzed descriptively by treatment group. Treatment comparisons are performed using Cochran-Mantel-Haenszel test controlling for the randomized stratum:
Percentage of subjects at Week 26 with normal ESR
Percentage of subjects at the end of randomized treatment with normal ESR
Percentage of subjects at Week 26 with normal CRP
Percentage of subjects at the end of randomized treatment with normal CRP
   The following continuous secondary efficacy endpoints are analyzed descriptively by treatment group. The analyses will include two-sided 95% CIs for the difference of treatment means, as appropriate:
Time to steroid dose of zero
Cumulative steroid dose at Week 26 and at the end of the double-blind treatment period
Change in clinical GCA assessments (including NRS and FACIT) over time
Change in quality-of-life over time
   The same approach is used for the following exploratory endpoint:
Reduction of vessel wall inflammation on biopsy (in consenting subjects) or imaging at Week 26

During the study, all adverse events and severe adverse events are followed until resolution. In case a suspicion of a flare/relapse, the Investigator is required to consult the Contract Research Organization (CRO)-designated Medical Expert to review and harmonize the elements of the diagnostic work-up. Flare/relapse is defined as a re-increase of CRP from normal to 1 mg/dL or greater and/or of ESR from less than 20 mm in the first hour to 30 mm or greater AND at least one of the following signs or symptoms attributed by the Investigator to new, worsening, or recurrent GCA:

### Cranial Symptoms:

New or recurrent headache or pain or tenderness of the scalp or the temporal artery
Visual signs/symptoms such as ischemic retinopathy, optic neuropathy, diplopia, amaurosis fugax, etc.
New or recurrent claudication of the tongue, masseter muscle, or worsening temporal artery signs and symptoms
Transient ischemic attack (TIA) or stroke related to GCA in the opinion of the Investigator

### Extracranial Symptoms:

Classic PMR-like symptoms, defined as shoulder and/or hip girdle pain associated with inflammatory morning stiffness
New or recurrent claudication in the peripheral circulation (i.e., in one of the extremities)
New or worsening angiographic abnormalities detected via MRI, CT/CTA, or PET-CT of the aorta or other great vessels or via ultrasound of the temporal arteries.

Supportive findings could include other symptoms in the opinion of the Investigator related to worsening GCA, such as sustained daily recurrent fever with a temperature over 38° C. for more than 1 week, chronic anemia, or unexplained weight loss.

All elements of the diagnostic work-up pertinent to the Investigator diagnosis of a flare/relapse (i.e., the primary clinical endpoint) should be reviewed with the CRO-designated Medical Expert and entered into the electronic Case Report Form (eCRF) promptly.

Flare/relapse is defined as major if cranial symptoms or ischemia-related visual loss are present, or if there is clear evidence of new onset large vessel vasculitis (e.g. subclavian artery). In all other situations flare/relapse attributed to PMR, vascular or other symptoms should be regarded as minor.

Cases of flare are treated according to the Investigator's judgment and standard of care (SoC) to ensure the best possible care of the subject. In general, the subject should continue to receive the assigned mavrilimumab or placebo and should also receive an increased dose of co-administered prednisone, as determined by the Investigator, generally of up to 60 mg/day. The dosages of all concomitant medications used to treat the GCA flare must be entered into the eCRF. If a flare, particularly a major flare, should require a dose of corticosteroid higher than prednisone 60 mg/day, in the judgment of the Investigator, steroid escape therapy is allowed (i.e., doses of prednisone >60 mg/day or equivalent, or IV corticosteroids) until clinical remission is achieved.

### Phase 2, Randomized, Double-Blind Placebo-Controlled Study to Test the Efficacy and Safety of Anti-GM-CSFRα Antibody in GCA

A global, multi-center, Phase 2, randomized, placebo-controlled Proof-Of-Concept study was designed to evaluate efficacy and safety of the anti-GM-CSFRα antibody (Mavrilimumab) with a 26-week corticosteroid (CS) taper in GCA subjects. Approximately 60 subjects aged 50-85 years with unequivocal signs and/or symptoms of GCA (cranial/extracranial), erythrocyte sedimentation rate >30 mm/hour or C-reactive protein ≥1 mg/dL, and diagnosis of GCA via temporal artery biopsy or imaging will be stratified by new onset or relapsing/refractory disease and randomized (3:2 ratio) to 150 mg anti-GM-CSFRα antibody or placebo administered subcutaneously every two weeks. Subjects receive mavrilimumab or placebo for 26 weeks (unless a subject discontinues treatment prematurely).

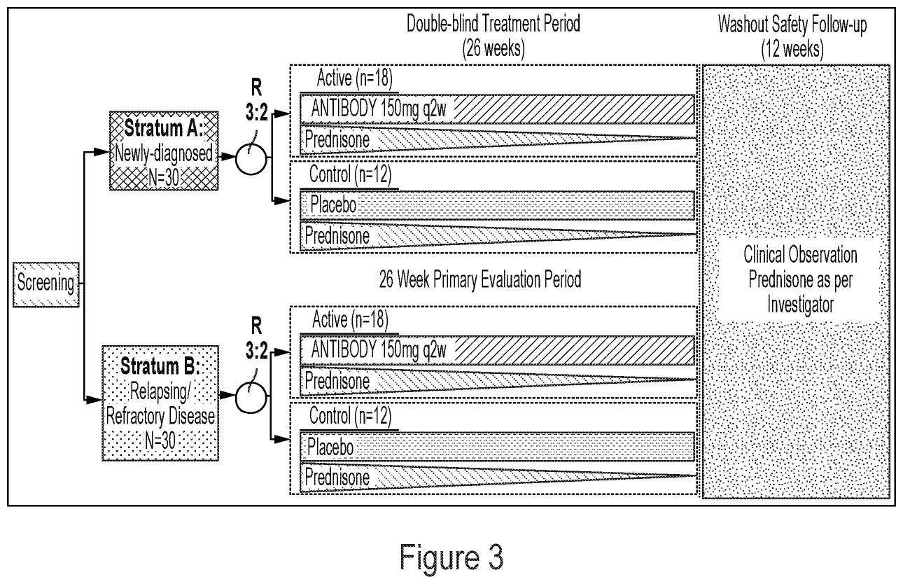

The primary efficacy endpoint is time to GCA flare (defined above). Secondary endpoints include time to CS dose of 0 mg/day, cumulative CS dose at Week 26 and at end of Washout Safety Follow-up, change in clinical GCA assessments, and change in quality-of-life. Safety measurements include incidence of adverse events, clinical laboratory variables, and pulmonary monitoring. FIG. 3 demonstrates a schematic outline of the study. A detailed description of the endpoints are provided above.

### Example 2: GM-CSF Pathway Signature in Temporal Artery Biopsies Giant Cell Arteries Biopsies

Two independent sources of temporal artery biopsies were utilized. First, GCA (n=18) and control (n=5) biopsies were analyzed for 5 mRNA transcripts representing T_{H}1, T_{H}17, and GM-CSF signaling (RNAscope; RS). Semi-quantitative scoring was performed on RS images of representative T_{H}1, T_{H}17 and GM-CSF related mRNA transcripts. Additional GCA and control biopsies were obtained and analyzed by RT-PCR for a subset of GM-CSF- and T_{H}1-associated transcripts (described further in Example 3). Additional GCA (n=3) and control (n=3) biopsies were obtained and GM-CSF and GM-CSFRα protein levels were detected by immunofluorescence and analyzed by confocal microscopy.

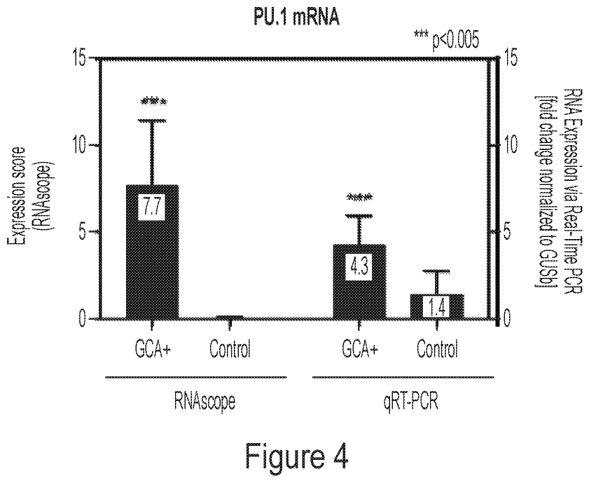

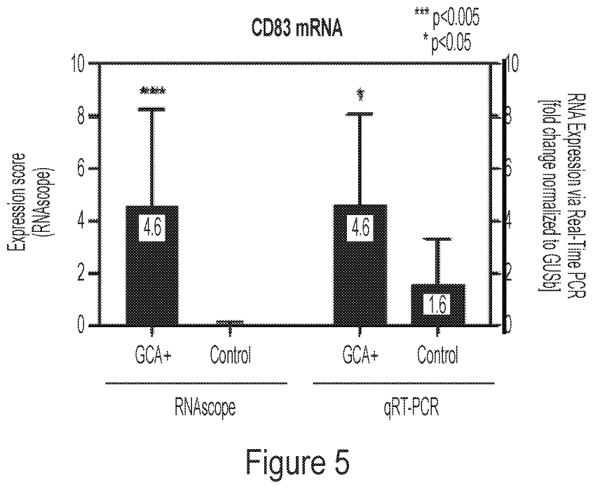

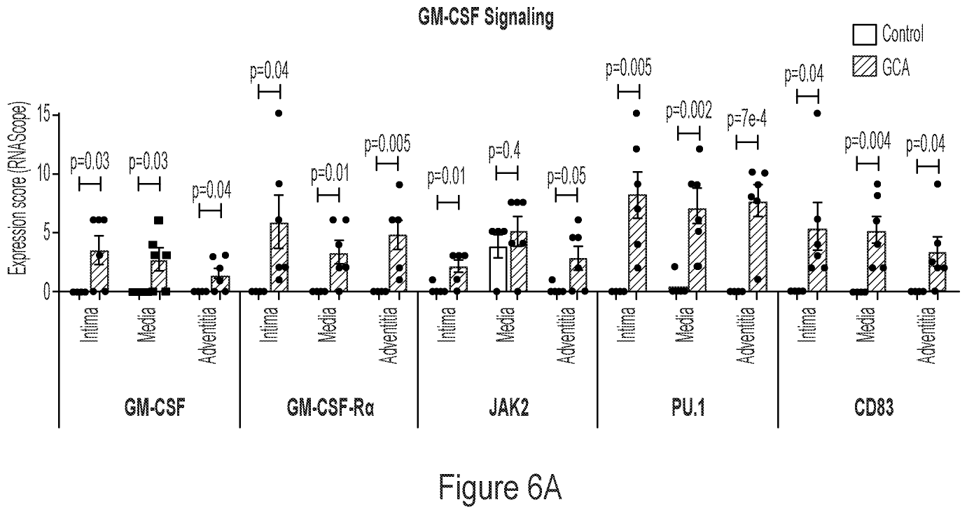

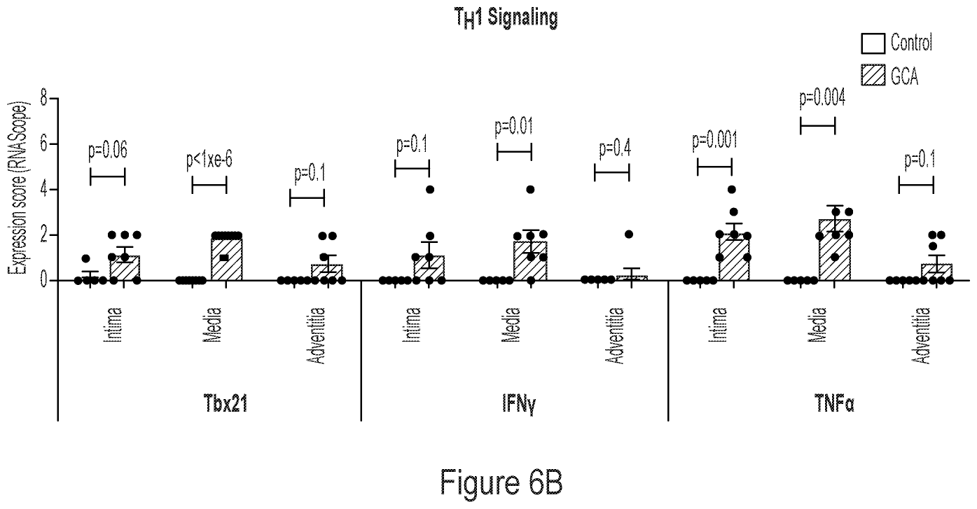

Expression of GM-CSF and GM-CSFRα mRNA as well as expression GM-CSF signaling- and T_{H}1-associated genes—was shown to be upregulated in GCA biopsies versus control. T_{H}17 associated genes were not elevated (data not shown), likely due to concomitant corticosteroid treatment. As shown in FIG. 4, Pu.1, a transcription factor downstream of GM-CSF signaling, was increased in GCA biopsies vs. controls (RS, RT-PCR). Increased levels of PU.1 protein localized to the nuclei (indicating activation of this transcription factor) was also observed in GCA arteries compared to control arteries by immunohistochemistry staining (data not shown). As shown in FIG. 5, CD83 mRNA was also upregulated in GCA biopsies vs controls (RS, RT-PCR). Expression levels of GM-CSF- and T_{H}1-associated genes (RS) across all three layers of the temporal artery vessel wall was determined in biopsies from GCA positive subjects and in biopsies from GCA negative (control) subjects. As shown in FIG. 6A, mRNA levels of GM-CSF-associated genes were upregulated in GCA biopsies (shaded bars) vs control biopsies (open bars). As shown in FIG. 6B, mRNA levels of T_{H}1-associated genes were upregulated in GCA biopsies (shaded bars) vs control biopsies (open bars)

### Example 3: GM-CSF and GM-CSF Receptor Expression Analysis from Giant Cell Arteries Biopsies by RT-PCR and Immunofluorescence

In this exemplary study the expression levels of GM-CSF mRNA, GM-CSFRα mRNA and INF-γ mRNA in the temporal artery biopsies were investigated in GCA patient samples relative to control samples.

Giant cell arteritis is understood to be predominantly a monocyte and macrophage related disease, and that GCA pathology could be associated with a higher expression of GM-CSF and its receptor. GM-CSF signaling helps induce monocyte-macrophage chemotaxis and activation. INF-γ is a signature cytokine produced by the Th1 cell lineage and has been implicated in multinucleated giant cell formation by promoting clustering and cell-to-cell adhesion. Expression of GM-CSF, GM-CSF receptor alpha (GM-CSFRα) and INF-γ transcripts were measured in the study described below.

Frozen Human temporal artery sections from either GCA patients (n=10) or control subjects (subjects without the disease) (n=10) were homogenized in TRIzol, and RNA was extracted using routine methods. mRNA was reverse transcribed to cDNA using random hexamer priming archive kit (Applied Biosystems, Foster City, Calif.). Real-Time Polymerase Chain Reaction (RT-PCR) was performed using Taqman probes (Applied Biosystems) specific for detecting GM-CSF, GM-CSFRα, INF-γ and GUSb. GM-CSF, GM-CSFRα or INF-γ gene expression was normalized to the expression of the endogenous control GUSb for each sample using comparative ΔCt method and was expressed in relative units with respect to GUSb expression.

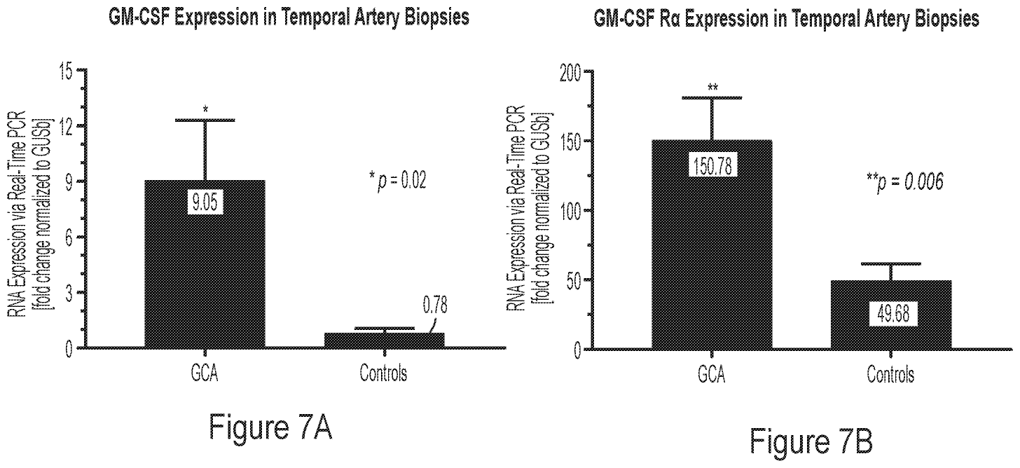

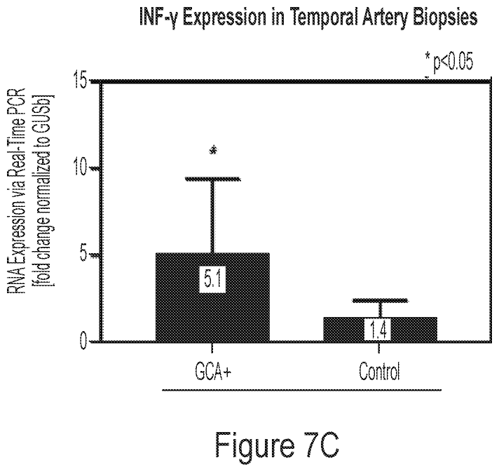

As shown in FIGS. 7A and 7B, GM-CSF and GM-CSFRα expression were substantially higher in GCA samples relative to the control. These data provide support that the GM-CSF pathway plays a significant role in GCA pathology and suggest that inhibition of the GM-CSF pathway by the receptor antagonist of the invention could positively impact the disease outcome. Similarly, as shown in FIG. 7C, INF-γ expression was elevated in GCA samples relative to control samples.

Immunofluorescence analysis of temporal lobe arteries obtained from GCA patients and control subjects who do not have GCA was performed to assess the presence and localization of GM-CSF and GM-CSFRα. The data obtained from the immunofluorescence analysis indicate that GM-CSFα was expressed on luminal endothelium in non-inflamed control biopsies as well as in GCA arteries, but expression was elevated in GCA arteries compared to controls. While GM-CSF is virtually absent in control arteries, it is widely expressed across inflamed arterial wall of GCA arteries. The data further indicate that GM-CSF and GM-CSFRα are both present in GCA lesions. Moreover, the immunofluorescence analysis revealed the presence of infiltrated macrophages near the media layer of the inflamed GCA artery that are positive for both GM-CSF and CD68 markers.

Activation of the GM-CSF and T_{H}1 pathways in temporal arteries of GCA patients was demonstrated by independent analytical techniques. Furthermore, active GM-CSF signaling in diseased tissue was evidenced by increased expression of Pu.1 in the vessel wall. These data implicate the GM-CSF pathway in GCA pathophysiology and further support the treatment of GCA by administering to a patient in need of treatment a GM-CSFRα antagonist, such as, for example, mavrilimumab.

### Example 4: GCA Artery Gene Expression Following Exposure to Mavrilimumab

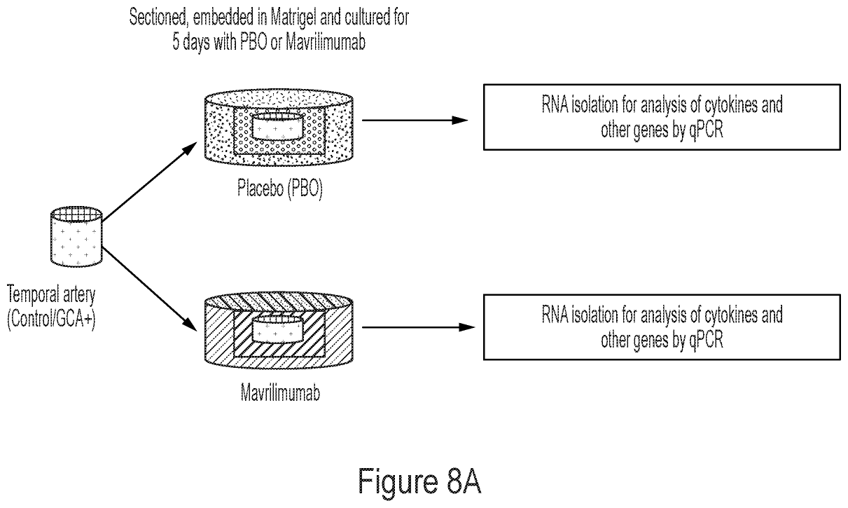

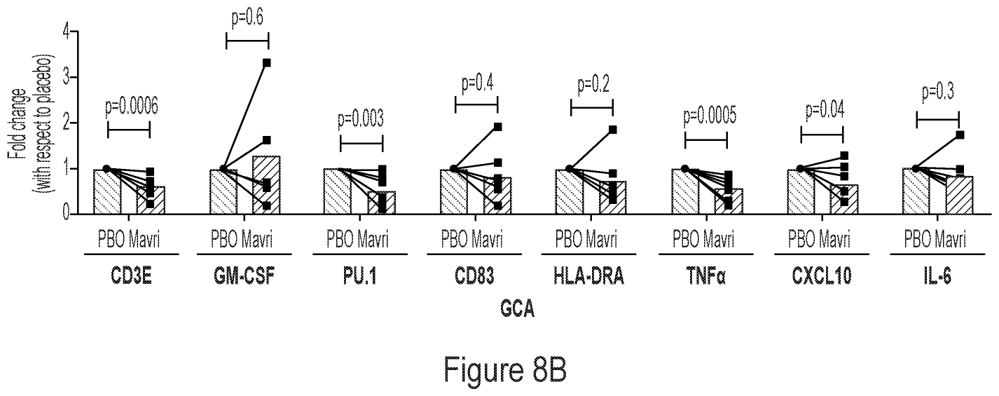

Temporal arteries from subjects who have GCA and from subjects who do not have GCA (control) were isolated, sectioned, embedded in Matrigel and cultured in the presence of either placebo or mavrilimumab. An established protocol was used for the temporal artery cell culture, and is described in detail in Corbera-Bellalta et al., Ann Rheum Dis., 2014:73:616-623, the contents of which is incorporated herein by reference in its entirety. A schematic that depicts temporal artery culture conditions used are presented in FIG. 8A.

The isolated arteries were cultured as described above in the presence of placebo or mavrilimumab for a period of 5 days. After the culture period, the arteries were processed for mRNA expression analysis. Treatment of ex vivo GCA artery cultures with mavrilimumab suppressed expression of inflammatory genes shown to be elevated in GCA, including CD3E, CD83, HLA-DR, TNFα, and CXCL10 (a chemokine secreted in response to INF-γ), indicating the biological effect of mavrilimumab on genes relevant to GCA pathophysiology. (FIG. 8B). These data clearly indicate that mavrilimumab reduces the expression of genes associated with GCA.

### Example 5: Human Temporal Artery Biopsies Engrafted into GCA Mouse Chimera Model

The human artery-NSG mouse chimera model was used to evaluate the efficacy of an anti-GM-CSFRα antibody (mavrilimumab) to suppress vessel inflammation and remodeling that occurs in vasculitic arteries. The human artery-NSG mouse chimera model used in this Example was previously described in detail in Zhang et al., Circulation, 2018:137(18):1934-1948. Briefly, normal temporal or axillary arteries were engrafted into NSG immune deficient mice. PBMCs from GCA patients were then adoptively transferred into the chimeric mice. About 7-10 days later, vasculitis of the engrafted human arteries was evident with tissue-infiltrating cells populating the vessel wall lesions. Tissue sections from the explanted human arteries demonstrated dense cell infiltrates. No vasculitis was observed if PBMCs from normal human controls are transferred. Interestingly, when 50 μg of recombinant GM-CSF (rGM-CSF) was administered to such chimeric mice, tissue inflammation in the arteries was intensified. The number of tissue-residing T cells doubled after rGM-CSF injection. The increase in the density of inflammatory cells was accompanied by parallel increase in the tissue gene expression of IL-1β, IL-6 and IFN-γ.

### Example 6: In Vivo Efficacy of Anti-GM-CSFRα Antibody in Treating GCA

In this Example, in vivo efficacy of mavrilimumab, a GM-CSF antagonist, in treating GCA was evaluated in the human artery-NSG mouse chimera model described above. For each group of mice, control IgG antibody or anti-GM-CSFRα antibody was administered intraperitoneally during established vasculitis (day 7 post adoptive transfer of GCA PBMCs). In this experiment, vasculitis in the chimeric mice was induced solely by adoptive transfer of PMBC's from GCA patients; no rGM-CSF was administered to the mice. Treating the chimeric mice at day 7 mimics treatment of steady-state vasculitis. In each experiment, mice were engrafted with segments from the same artery and received an adoptive transfer of PMBCs from the same patient, so that the vasculitis was comparable in each of the treatment arms. Following a 1-week treatment period, arteries were harvested and examined by immunohistochemistry and transcriptome analysis.

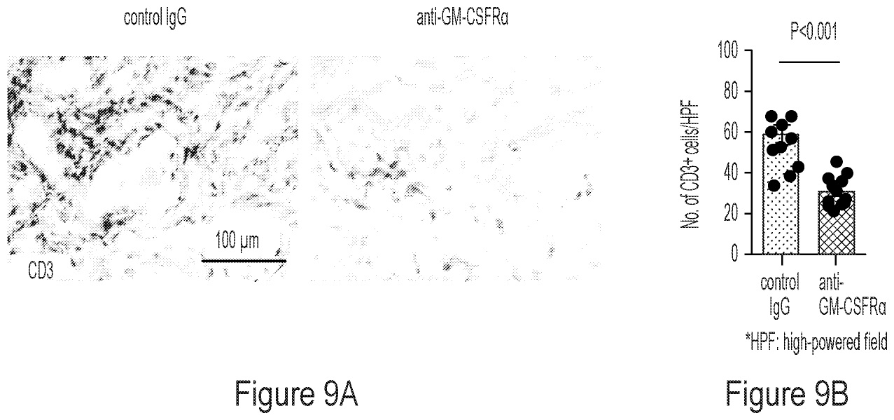

As depicted in FIG. 9A, immunohistochemistry staining for CD3⁺ T cells show that tissue-infiltrating CD3⁺ T-cells were significantly reduced in mice administered with anti-GM-CSFRα antibody as compared to mice administered with IgG control antibody. T-cell-depleting effect was also examined by enumerating T-cell counts in the inflamed artery tissue. As shown in FIG. 9B, the number of tissue-residing T-cells per high-powered field was about 50% lower in anti-GM-CSFRα antibody-treated mice as compared to IgG control-treated mice (with statistical significance of P<0.001). These data illustrate that treating the chimeric mice with anti-GM-CSFRα antibody showed strong anti-inflammatory effects compared to the isotype antibody negative control arm.

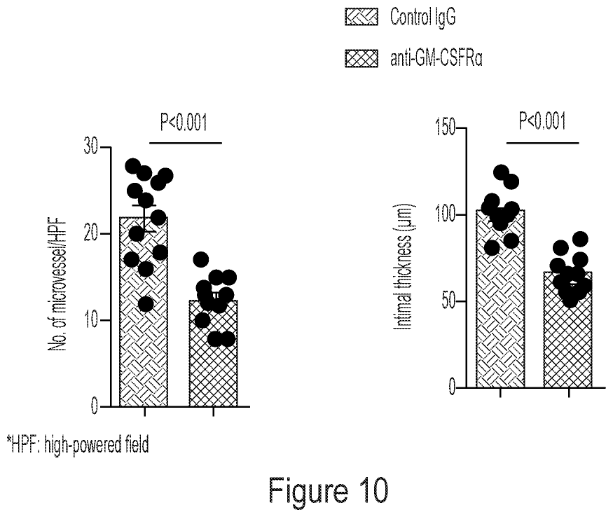

When affected by GCA, large and medium arteries develop a dense network of microvessels, resulting in neoangiogenesis. T-cells in arteries also promote intimal hyperplasia, measured by thickness of the intimal (innermost) layer of the arteries. As shown in FIG. 10, the number of microvessels in anti-GM-CSFRα-treated mice was significantly reduced as compared to the control IgG-treated mice. Moreover, the intimal thickness measurements fell by about 40% (with statistical significance of P<0.001) in mice that were administered anti-GM-CSFRα antibody, as compared to mice that were administered IgG control antibody. These results illustrate that the density of the inflammatory infiltrates in mice treated with anti-GM-CSFRα was suppressed and the wall remodeling process was inhibited.

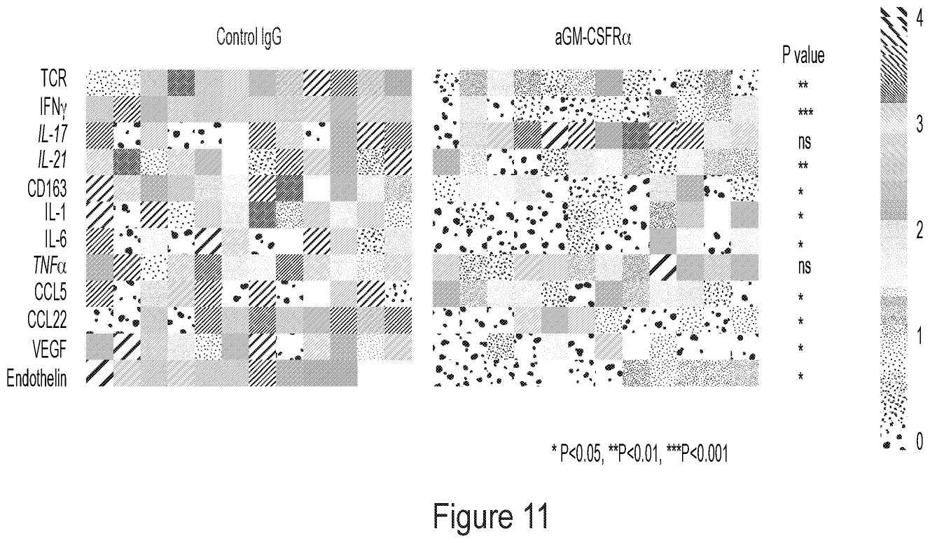

Next, the gene expression profile in the artery tissue of IgG control- or anti-GM-CSFRα-treated mice was evaluated and plotted as a heatmap, where rows represent genes, and columns represent mice. FIG. 11 shows that tissue transcriptome for proinflammatory cytokines in the IgG control-treated mice was significantly elevated compared to that of anti-GM-CSFRα-treated mice. For example the tissue gene expression of IL-1β, IL-6, and IFN-γ was significantly reduced in mice treated with anti-GM-CSFRα antibody. The reduced expression of IFN-γ in mice treated with anti-GM-CSFRα antibody is of particular significance for treating GCA as it is the signature cytokine produced by the Th1 cell lineage (a cell lineage with vasculitogenic potential) and has been implicated in multinucleated giant cell formation by promoting clustering and cell-to-cell adhesion. Further, IFN-γ-producing Th1 cells are relatively unresponsive to glucocorticoid therapy and persist in steroid-treated patients, and overproduction of IFN-γ is believed to be a critical mechanism in the chronicity of the disease. These results illustrate that anti-GM-CSFRα antibody can suppress innate and adaptive immune response in the inflamed artery.

Overall, these in vivo data suggest that administration of an GM-CSF antagonist (e.g., an anti-GM-CSFRα antibody) can be used to treat vascular inflammation, intimal hyperplasia, and neoangiogenesis, which are key aspects of GCA pathology.

### EQUIVALENTS

Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

1. A method of treating giant cell arteritis (GCA), comprising administering to a subject in need of treatment a composition comprising a granulocyte-macrophage colony-stimulating factor (GM-CSF) antagonist.

2. The method of claim 1, wherein the GM-CSF antagonist is a GM-CSF receptor antagonist.

3. The method of claim 2 wherein the GM-CSF receptor antagonist is an antibody specific for human GM-CSFRα.

4. The method of claim 3, wherein the anti-GM-CSFRα antibody is mavrilimumab

5. The method of claim 1, wherein the GM-CSF antagonist is an antibody specific for GM-CSF.

6. The method of claim 5, wherein the anti-GM-CSF antibody is namilumab, otilimab, gimsilumab, lenzilumab or TJM-2.

7. The method of any one of preceding claims, wherein the subject is between 50 and 85 years of age.

8. The method of claim 1, wherein the giant cell arteritis is new-onset disease.

9. The method of claim 1, wherein the giant cell arteritis is relapsing disease.

10. The method of claim 1, wherein the giant cell arteritis is a refractory disease.

11. The method of any of the preceding claims, further comprising, co-administering a corticosteroid to a subject in need thereof.

12. The method of any of the preceding claims, wherein the dose of the co-administered corticosteroid is tapered over the course of the treatment with the GM-CSF antagonist.

13. The method of claim 1, wherein the treating results in the prevention, reduction or amelioration of at least one of the disease symptoms associated with GCA.

14. The method of claim 13, wherein the treating results in elimination of symptoms associated with GCA.

15. The method of claim 13 or 14, wherein the treating reduces arterial inflammation and/or reduces expression of genes associated with GCA lesions.

16. The method of claim 15, wherein the reduced expression of genes associated with GCA lesions results in reduced expression of protein and/or messenger RNA (mRNA) selected from GM-CSF, GM-CSFRα, JAK2, IL-6, CD83, PU.1, HLA-DRA, CD3E, TNFα, IL-1β, or combinations thereof.

17. The method of any one of claims 13-16, wherein the treating results in the reduction or elimination of infiltrated macrophages, reduced T-cells in vessel adventitia, reduced GM-CSFRα expression in vasa vasorum of the temporal artery, reduced density of inflammatory infiltrates, and/or reduced or stabilized vessel wall remodeling.

18. The method of any one of claims 13-17, wherein the treating results in a reduction of cells positive for GM-CSF or INF-γ in the arterial wall.

19. The method of any one of claims 13-18, wherein the treating normalizes gene expression levels comparable to a subject who does not have GCA.

20. The method of claim 19, wherein the treating normalizes gene expression levels of genes associated with interferon signaling, IL-6 signaling and/or GM-CSF signaling.

21. The method of claim 20, wherein the treating normalizes gene expression levels of genes associated with interferon signaling selected from INF-γ, INF-αR1, INF-γR1, INF-γR2, IFI30, IFI35, PRKCD, B2M, IFNAR1, CIITA, PTPN2, PTPN11, IRF1, IFR5, IRF8, GBP1, GBP5, STAT1, STAT2, FCγR1A/B, ICAM1, VCAM1, TYK2, CD44, IP6K2, DDX58, PTPN6, or combinations thereof.

22. The method of claim 20, wherein the treating normalizes gene expression levels of genes associated with IL-6 signaling selected from PTPN11, TYK2, STAT1, IL-11RA, IL-6, or combinations thereof.

23. The method of claim 20, wherein the treating normalizes gene expression levels of genes associated with GM-CSF signaling selected from IL-2RB, IL-2RG, GM-CSFRα, JAK3, STAT5A, SYK, PTPN11, HCK, FYN, INPP5D, BLNK, PTPN6, or combinations thereof.

24. The method of claim any of the preceding claims, wherein the at least one of the disease symptoms associated with giant cell arteritis comprise fever, fatigue, weight loss, headache, temporal tenderness, and jaw claudication; transient monocular visual loss (TMVL) and anterior ischemic optic neuropathy (AION), aortic aneurism and vasculitis.

25. The method of claim 1, wherein the subject has a serum inflammatory marker CRP ≥1 mg/dL prior to administering the composition.

26. The method of claim 1, wherein the composition comprising GM-CSF antagonist is administered at a dose of 150 mg.

27. The method of claim 1, wherein the composition comprising GM-CSF antagonist is administered once every two weeks.

28. The method of claim 4, wherein mavrilimumab is administered by intravenous or subcutaneous administration.

29. The method of any one of the preceding claims, wherein the subject is co-administered an additional therapeutic agent.

30. The method of claim 29, wherein the additional therapeutic agent is a corticosteroid.

31. The method of claim 30, wherein the corticosteroid is prednisone.

32. The method of claim 11 or 12, wherein the additional therapeutic is a co-administered corticosteroid that is tapered over 26 weeks.

33. The method of any one of the preceding claims, wherein administering the composition comprising GM-CSF antagonist reduces serum inflammatory marker CRP to <1 mg/dL.

34. The method of any one of the preceding claims, wherein administering the composition comprising GM-CSF antagonist reduces ESR ≤30 mm/hour.

35. The method of any one of the preceding claims, wherein administering the composition comprising GM-CSF antagonist results in sustained remission of symptoms associated with GCA.

36. The method of claim 35, wherein the remission is sustained with a reduction of co-administered corticosteroids.

37. The method of claim 36, wherein the sustained remission is substantially corticosteroid-free.

38. The method of claim 37, wherein the sustained remission is corticosteroid free.

39. The method of any one of the preceding claims, wherein administering the composition comprising GM-CSF antagonist results in patients achieving a sustained remission for 26 weeks.

40. The method of claim 3, wherein the anti-GM-CSFRα antibody comprises a light chain complementary-determining region 1 (LCDR1) defined by SEQ ID NO: 6, a light chain complementary-determining region 2 (LCDR2) defined by SEQ ID NO: 7, and a light chain complementary-determining region 3 (LCDR3) defined by SEQ ID NO: 8; and a heavy chain complementary-determining region 1 (HCDR1) defined by SEQ ID NO: 3, a heavy chain complementary-determining region 2 (HCDR2) defined by SEQ ID NO: 4, and a heavy chain complementary-determining region 3 (HCDR3) defined by SEQ ID NO: 5.


SEQUENCE LISTING (8 sequences)

SEQ ID NO: 1 - protein, 120 aa, Artificial Sequence
  Synthetic polypeptide
QVQLVQSGAEVKKPGASVKVSCKVSGYTLTELSIHWVRQAPGKGLEWMGGFDPEENEIVYAQRFQGRVTMTEDTSTDTAYMELSSLRSEDTAVYYCAIVGSFSPLTLGLWGQGTMVTVSS

SEQ ID NO: 2 - protein, 111 aa, Artificial Sequence
  Synthetic Polypeptide
QSVLTQPPSVSGAPGQRVTISCTGSGSNIGAPYDVSWYQQLPGTAPKLLIYHNNKRPSGVPDRFSGSKSGTSASLAITGLQAEDEADYYCATVEAGLSGSVFGGGTKLTVL

SEQ ID NO: 3 - protein, 5 aa, Artificial Sequence
  Synthetic oligopeptide
ELSIH

SEQ ID NO: 4 - protein, 17 aa, Artificial Sequence
  Synthetic oligopeptide
GFDPEENEIVYAQRFQG

SEQ ID NO: 5 - protein, 11 aa, Artificial Sequence
  Synthetic oligopeptide
VGSFSPLTLGL

SEQ ID NO: 6 - protein, 14 aa, Artificial Sequence
  Synthetic oligopeptide
TGSGSNIGAPYDVS

SEQ ID NO: 7 - protein, 7 aa, Artificial Sequence
  Synthetic oligopeptide
HNNKRPS

SEQ ID NO: 8 - protein, 11 aa, Artificial Sequence
  Synthetic oligopeptide
ATVEAGLSGSV